(19) United States
(12) Patent Application Publication
(10) Pub. No.: US 2026/0325146 A1
(43) Pub. Date: Oct. 22, 2026

(54) SPINAL STIFFNESS SYSTEMS AND RELATED METHODS

(71) Applicant: GLOBUS MEDICAL, INC., AUDUBON, PA (US)

(72) Inventors: David C. Paul, Phoenixville, PA (US); Patrick Murray, Collegeville, PA (US); Nicholas Meadows, Langhorne, PA (US); George Yacoub, Lansdale, PA (US)

(21) Appl. No.: 19/723,646

(22) Filed: Jun. 29, 2026

Related U.S. Application Data
(62) Division of application No. 18/645,517, filed on Apr. 25, 2024.

(51) Int. Cl. A61B 17/17; A61B 17/02; A61B 17/70; A61B 17/00; A61B 90/00

(52) U.S. Cl. CPC A61B 17/1757; A61B 17/025; A61B 17/7086; A61B 2017/00407; A61B 2017/0256; A61B 2090/064; A61B 2090/065

(57) ABSTRACT

Devices, systems, and methods for evaluating spinal stiffness of a patient. One method may include providing a database model based on existing patient data with normalized spine stiffness data. Segmental stiffness may be measured intraoperatively, for example, using a force-sensing instrument, and compared to the database model. A surgical task, such as osteotomy or ligament release, may be performed based on guidance from the database model to adjust the spinal stiffness of the patient. Segmental stiffness may be measured after each surgical task, thereby updating the database model with each reading on segmental stiffness in real time. Each level may be addressed until targeted stiffness values, such as segmental stiffness and global stiffness, are reached based on the database model.

### CROSS REFERENCE TO RELATED APPLICATIONS

The present application is a divisional application of U.S. Patent Application Serial No. 18/645,517, filed on Apr. 25, 2024, which is incorporated in its entirety herein.

### FIELD OF THE INVENTION

The present disclosure relates to spinal stiffness systems, and more particularly, to utilizing force-sensing instruments and spinal stiffness database models to optimize surgical outcomes.

### BACKGROUND OF THE INVENTION

Many types of spinal irregularities cause pain, limit range of motion, or injure the nervous system within the spinal column. These irregularities may result from, without limitation, trauma, tumor, disc degeneration, disease, and deformity. Often, these irregularities are treated by immobilizing a portion of the spine, for example, by affixing screws to the vertebrae and connecting the screws to an elongate spinal rod that stabilizes the spine.

During spine surgery, the surgeon may manually apply forces and moments to correct the spine. These corrective forces may be exerted on the spine via instruments and/or implants attached to the spine. Spine stiffness is an important parameter that surgeons may consider when surgically correcting spinal deformities. The amount of force needed to correct the spine deformity varies from patient to patient. A patient with a high spine stiffness may require more corrective force than a patient with a low spine stiffness. Spine stiffness may be modified during surgery, for example, by performing osteotomies or ligament releases. These actions, however, may cause blood loss, instability, and additional morbidity. Therefore, surgeons may balance factors between loosening the spine enough to achieve correction and minimizing negative disruption to the anatomy. Current methods of assessing patient spine stiffness may include preoperative imaging studies, such as side bending or fulcrum bending tests, physical tests such as push-prone tests, traction, and push-traction. Spine stiffness is often misjudged preoperatively such that the stiffness of a patient’s spine may be more or less stiff during the operation than the clinician determined preoperatively. This may cause changes to the surgical plan, adding time and stress to the procedure.

The introduction of robotics into spine surgery has enhanced safety and improved efficiency for surgeons during deformity correction. Imaging and navigation technologies combined with robotics have enabled surgeons to receive real time feedback on clinically significant parameters that previously could not be assessed intraoperatively. There exists a need, however, for devices and methods of improving feedback and information to the surgeons. Devices and methods integrating robotic, imaging, and/or navigation technologies into spinal deformity correction procedures may further improve the safety, efficacy, reliability, and/or repeatability of correction maneuvers during deformity surgery.

### SUMMARY OF THE INVENTION

To meet this and other needs, spinal stiffness systems, force-sensing instruments, and related methods are provided. In order to assess the true mechanical stiffness of a patient’s spine, spinal stiffness systems and methods may include intraoperative measurements of spine stiffness on individual spine segments. For example, force-sensing instruments may be configured to measure both displacement and force, which provide the surgeon with a more accurate understanding of the patient’s specific spinal biomechanics during surgery. The data from the force-sensing instrument may be used to calculate the forces and moments exerted on the patient to allow the surgeon to perform a more appropriate level of intervention. In addition, the spinal stiffness systems and methods may incorporate a comprehensive database model based on extensive data inputs with outputs for the specific patient profile. The database model may include spine stiffness data collected from the patient, in real time, during an operation, such as a lumbar decompression. The decompression may be performed sequentially for each level and the database model may be updated with each spinal stiffness reading, thereby providing a real-time feedback loop. The database model may incorporate artificial intelligence or machine learning, for example, to enhance data analysis and predictions. The force-sensing instruments and database model may be incorporated into computer-assisted technology platforms, such as robotic and/or navigation systems to further assist the surgeon throughout the surgical procedure.

According to one embodiment, a method of evaluating spinal stiffness for a spine of a patient may include: (a) providing a database model based on existing patient data with normalized spine stiffness data; (b) measuring segmental stiffness of a motion segment between two vertebrae of the spine of the patient intraoperatively; (c) comparing the measured segmental stiffness to the database model and estimating how much the vertebrae will move based on the model; (d) performing a surgical task based on guidance from the database model to adjust the spinal stiffness; and (e) measuring segmental stiffness after the surgical task and updating the database model with each reading on segmental stiffness in real time. Steps (b)-(e) are repeated for each level until targeted stiffness values are reached based on the database model.

The method of evaluating spinal stiffness may include one or more of the following features. The guidance from the database model may include expected values for the patient in their current condition and expected values for the patient after correction. The database model may provide the normalized spine stiffness data for each level and global stiffness values. Each level of the spine may have its own segmental stiffness value, which is variable depending on the patient. The surgical task may include an osteotomy or ligament release to decrease segmental stiffness. The database model may identify how and where osteotomies are needed including the number and size of the osteotomies. The database model may incorporate artificial intelligence to enhance database functionality, data analysis, and predictions. The database model may be incorporated into software of an on-board computer for a surgical robotic and navigation system.

According to one embodiment, a method of correcting a spinal deformity of a patient may include: (a) applying a force to a spine having a deformity with a force-sensing instrument to measure spine stiffness; (b) comparing the measured spine stiffness to a database model with existing spinal stiffness parameters; (c) obtaining guidance from the database model based on patient specific parameters for the patient; (d) performing a decompression sequentially on the spine, based on the guidance from the database model; (e) measuring spine stiffness throughout the decompression and updating the database model with each reading on spine stiffness in real time; (f) obtaining a correction of the deformity when targeted stiffness values are reached based on the database model; and (g) finalizing the deformity correction by installing spinal hardware.

The method of correcting a spinal deformity may include one or more of the following features. The existing spinal stiffness parameters may include segmental stiffness, stiffness across motion segments, and/or global stiffness values. The existing spinal stiffness parameters may include averaged or normalized spine stiffness values. The existing spinal stiffness parameters may be based on inputs of publicly available data including demographic data and clinical data. The clinical data may include spine stiffness data for intact spines, spines having a deformity, and spines having underwent a prior correction. The database model may include data aggregated into ranges. The force-sensing instrument may measure segmental stiffness of a single motion segment between two vertebrae.

According to one embodiment, a system for evaluating spinal stiffness for a spine of a patient may include a surgical robotic and navigation system having an on-board computer with software executed by one or more processing units, and storing and executing an existing database model with spine stiffness parameters, and a force-sensing instrument for measuring spine stiffness intraoperatively. The system compares measured spine stiffness to expected spine stiffness values from the database model and provides guidance to a surgeon during a procedure. During the procedure, the measured spine stiffness is added into the database model, updating the database model in real time, thereby providing a feedback loop with each measurement until desired spine stiffness values are reached from the database model.

The system for evaluating spinal stiffness may include one or more of the following features. In one embodiment, the force-sensing instrument may be a navigated spreader instrument with built-in force measuring, wireless communication, and navigation tracking. The navigated spreader instrument may include two pivotable arms connected by a hinge with distal tips configured to engage the spine, an electronics package around a sensing portion, a ratchet with a reflective marker, and a navigation array with reflective markers for instrument tracking by the navigation system. In another embodiment, the force-sensing instrument may be a rod link reducer with built-in force measuring and wireless communication. The rod link reducer may include a manipulating arm with a clamping portion sized to releasably retain a spinal rod therein. The manipulating arm may have a strain bridge with a strain gage to measure and analyze strain on the instrument and communicate to the software, which calculates the forces and moments exerted on the patient. The force-sensing instrument may include any suitable navigated or non-navigated instrument configured to measure spine stiffness, when forces are applied to the bones (e.g., vertebrae) or to the implants (e.g., bone screws or rods).

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 illustrates a diagrammatic view of a comprehensive spine stiffness database model based on extensive data inputs, such as publicly available demographic and clinical data, with outputs for a specific patient profile;

FIG. 2 is a schematic representation of the lordotic spine and the segmental stiffness for each spinal level;

FIG. 3 illustrates a flowchart for updating the database model with spine stiffness data collected from a patient, in real time, during an operation;

FIG. 4 illustrates a flowchart for optimizing a spinal procedure by comparing measured spine stiffness values during the procedure with expected values obtained from the database, making corrections based on the model, and iteratively updating the model throughout the procedure to achieve the desired outcome;

FIGS. 5A-5B show examples of surgical robotic and navigation systems;

FIGS. 6A-6B shows a force-sensing spreader instrument with built-in force measuring, wireless communication, and navigation tracking, and with the electronic package omitted to reveal the strain bridge, respectively;

FIGS. 7A-7B show close-up views of the strain bridge and a strain gage mounted to the strain bridge, respectively;

FIG. 8 is an exploded view of the electronic package and the spreader instrument;

FIG. 9 shows a cross-section of a tracking array attached to the spreader instrument and a single ratchet marker configured to track the distance that the distal tips spread apart;

FIG. 10 shows an operating room setup with the force-sensing spreader instrument trackable by the robotic and navigation system;

FIGS. 11A-11B show the spreader instrument in a closed condition (left) and an open condition (right), which produces a difference in distance between the ratchet marker and the tracking array according to one embodiment;

FIG. 12 depicts the force-sensing spreader placed between two vertebrae to determine segmental stiffness intraoperatively;

FIG. 13 depicts a schematic of the spine with a gradient to depict trends or changes in spine stiffness between levels, which may be displayed on a monitor of the surgical robotic and navigation systems for the surgeon;

FIG. 14 illustrates a method of simulating deformity correction using a kinematic model of the spine according to one embodiment;

FIG. 15 illustrates a method for creating a patient specific spinal rod intraoperatively using spine stiffness measurements and a kinematic model of the spine according to one embodiment;

FIG. 16 shows how the shape of a spinal rod may change during a deformity correction;

FIG. 17 shows a method for creating a patient specific spinal rod intraoperatively using bone screw interface strength, spine stiffness measurements, and a kinematic model of the spine according to one embodiment;

FIG. 18 is a graph of the relationship between screw insertion torque and pullout strength;

FIG. 19 shows a method for creating a patient specific spinal rod intraoperatively using bone density, bone screw interface strength, spine stiffness measurements, and a kinematic model of the spine according to one embodiment;

FIG. 20 shows a force-sensing rod link reducer instrument with built-in force measuring and wireless communication according to one embodiment;

FIG. 21 shows the force measuring rod link reducer instrument system attached to the spine to perform a deformity correction;

FIGS. 22A-22B show close-up views of single style (left) and double style (right) manipulating arms for the rod link reducer instrument;

FIG. 23 shows an exploded view of the rod link reducer instrument of FIG. 20;

FIGS. 24A-24B show a distal portion of the manipulating arm with a strain bridge and a strain gage attached to the strain bridge, respectively;

FIG. 25 shows an exploded view of the electronics package for the rod link reducer instrument;

FIG. 26 shows the force-sensing rod link reducer instrument system according to one embodiment;

FIG. 27 shows an operating room setup with the force-sensing rod link reducer system attached to a patient, which communicates force measurements to the robotic system;

FIG. 28 shows a navigated force-sensing rod link reducer instrument according to one embodiment;

FIG. 29 shows an operating room setup with the navigated force-sensing rod link reducer system attached to a patient and configured to communicate force measurements to a robotic and navigation system;

FIG. 30 illustrates a method of assessing spine stiffness intraoperatively with the navigated force measuring rod link reducer system; and

FIG. 31 illustrates a method of tracking deformity correction using a kinematic model of the spine, which is updated with the navigated force measuring rod link reducer system.

### DETAILED DESCRIPTION OF THE INVENTION

Embodiments of the disclosure are generally directed to spinal stiffness systems, force-sensing instruments, and related methods. In particular, a spinal stiffness information system may include a comprehensive database model, which stores, manages, and retrieves data, for example, relating to spinal stiffness parameters and other patient data. The comprehensive database may incorporate demographic data, clinical data, and other prior knowledge into the model. During a spinal procedure, force-sensing instruments may be used, for example, to apply forces before correction and/or to correct the spine in compression, distraction, reduction, and/or derotation. The force-sensing instrument may be used to characterize the stiffness of the spine due to the applied forces in real time. The information system may include algorithms configured to analyze the patient data for a given patient in relation to the stored and expected outcomes in the database. The system may compare the expected spine stiffness data to intra-operative data for the patient, which allows the surgeon to make informed decisions during the procedure. The procedure may proceed, for example, level by level, until the desired outcomes (e.g., segmental and global spinal stiffness values) are achieved. In an exemplary embodiment, the procedure may continue until optimal targets or ranges are reached from the database model. The targets may include a desired spinal stiffness, for example, for segmental stiffness, motion segments, or global stiffness. In addition, the database may be updated in real time based on the patient’s specific data measured during the procedure, thereby creating a real-time feedback loop. Although generally described for use with correcting a spinal deformity, it will be readily appreciated by those skilled in the art that the systems and methods described herein may be employed in any number of suitable orthopedic applications or other surgical procedures.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments or modifications thereof are contemplated as falling within the scope of this disclosure and equivalents thereto.

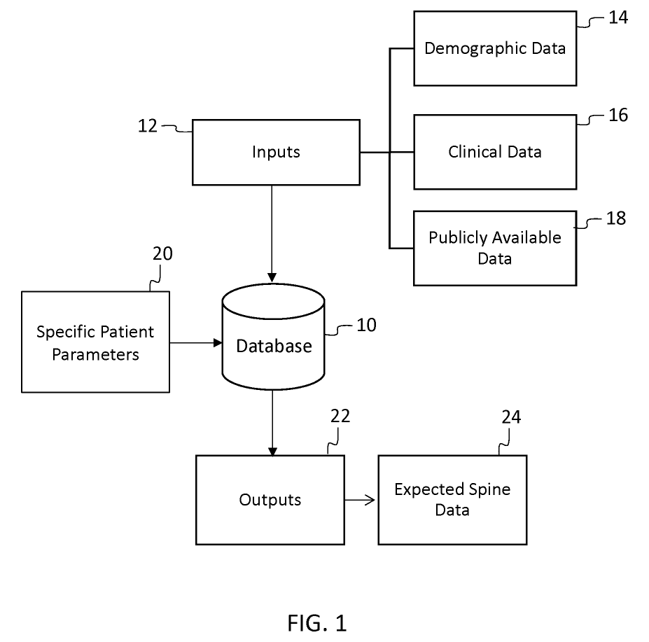

Turning now to the drawing, where like numerals indicate like elements throughout, FIG. 1 illustrates a comprehensive spine stiffness database model 10, which is configured to store, manage, and analyze existing patient data. The database 10 may encompass various entities (e.g., tables) to capture the breadth of existing patient information, clinical data, treatment history, outcomes, and the like. The database 10 may be comprised of inputs 12 obtained from a variety of sources. The database inputs 12 may include demographic information 14, such as age, gender, body weight, height, body mass index (BMI), race/ethnicity, smoking status, geographic location, or other relevant patient data. The inputs 12 may further include clinical data 16, such as spinal stiffness parameters (e.g., segmental stiffness, stiffness across motion segments, global stiffness values), vertebral body height or size, bone density, disc height or integrity, intact normal spines or spines having spinal misalignments (e.g., scoliosis, kyphosis, or spondylolisthesis), updated outcomes following a surgical procedure, such as a decompression procedure (e.g., updated stiffness values after laminectomy, facetectomy, osteotomies, supraspinous or interspinous ligament releases, discectomy, foraminotomy, corpectomy), or any other biomechanical data. The clinical data 16 may also include other patient data, such as blood pressure, heart rate, biomarker data (e.g., molecular or cellular markers), comorbidities or chronic conditions, mental state (e.g., anxiety, depression), allergies, pain type/level/duration (e.g., Oswestry Disability Index or Visual Analog Scale (VAS)), or other relevant health data.

The inputs 12 may be obtained from publicly available data 18, for example, including known and standard information from registries, clinical trials, government sources (e.g., National Institute of Allergy and Infection Diseases (NIAID)), hospitals, journals and publications, or other existing clinical databases or public data sets. The inputs 12 may also be collected from private sources, such as private hospitals, proprietary databases, or surveys. Any data collection practices comply with all relevant laws, such as healthcare regulations (e.g., HIPAA) and data protection regulations (e.g., GDPR). The data may be cleaned to correct inaccuracies, structured and organized into a coherent database structure with clear relationships between data sets, and stored based on the volume and type of data. The database 10 may be regularly updated with new data and by removing outdated information with security measures to protect the data against unauthorized access, breaches, or security threats.

The data points or data sets may be modeled to include averaged or normalized values to facilitate comparison and improve statistical analysis. The relevant data points may be aggregated over a defined period or category. The collected values may be averaged, and the calculated averages may be stored in the database, optionally alongside the raw data. The averaging or normalization statistics may include mean averages, weighted averages, log-normalized averages, high adjusted R-squared, lowest Akaike Information Criteria (AIC), lowest Bayesian Information Criteria (BIC)), model parsimony, min-max normalization, Z-score standardization, quantile normalization, robust scaling, decimal scaling, or any other suitable statistical method. The normalization may be used to adjust the scale of the data without distorting differences in the range of values, and may be useful for understanding the general performance or trend within a data set. In addition, the data may be aggregated into ranges or sets. For example, in the case of age, rather than analyzing individual ages, ages may be grouped into ranges such as 0-10 years, 11-18 years, 19-35 years, 36-50 years, 51-65 years, and older than 65. This reduces the complexity by categorizing numerous individual data points into broader, more manageable groups. The database 10 provides a robust foundation for storing, managing, and utilizing patient information, and as described in more detail below, is configured to support and guide a surgeon while performing a surgical task, such as spinal decompression.

A user may access the database 10 by providing specific patient parameters 20 for a given patient to obtain one or more outputs 22 from the database 10. For example, the user may enter parameters 20, such as a patient’s age, gender, and medical condition (e.g., spondylolisthesis) to obtain outputs 22 on expected spine data 24, such as expected stiffness for a given vertebral level, motion segment, or global spine stiffness. The expected spine data 24 may include expected values for the patient in their current condition (e.g., spondylolisthesis), expected values for the patient after correction (e.g., following decompression), and/or expected values for a normal intact spine. In this manner, analyses and comparisons may be made between current and future expected values for a given patient for planning, during the surgery, and to improve patient outcomes.

In one embodiment, the database model 10 may incorporate artificial intelligence (AI) to enhance database functionality with AI algorithms and machine learning (ML) models, enabling advanced data analysis and predictions. The AI algorithms may include supervised learning, unsupervised learning, semi-supervised, and reinforcement learning algorithms. The AI database 10 may process and analyze large volumes of data, extract insights, predict trends, and learn from new data inputs over time to optimize outcomes. For example, the AI database 10 may understand and optimize complex queries, thereby providing faster and more accurate responses. The AI database 10 may analyze historical data to predict trends or potential pitfalls. AI-driven automation may handle routine data management tasks, such as indexing, backups, and data integrity checks. The AI database 10 may continuously monitor its performance and automatically adjust or reorganize data to optimize performance. The AI database 10 may identify patterns, anomalies, and correlations within the data that may not be otherwise apparent. It will be appreciated that any suitable AI algorithms or machine learning models may be used based on the most appropriate methodologies.

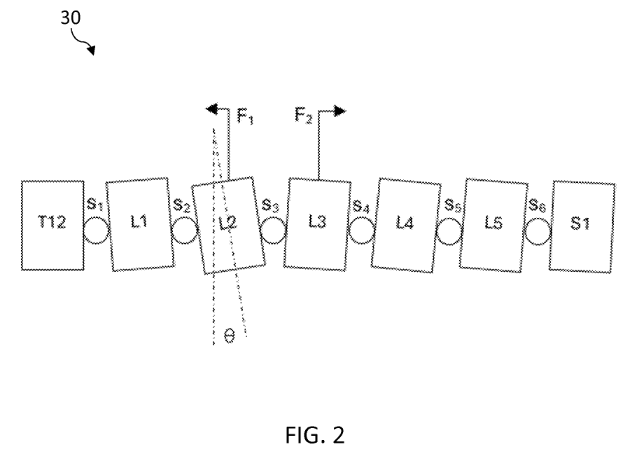

Turning now to FIG. 2, a schematic representation of a portion of the spine 30 is shown. The lumbar spine 30 includes the lower end of the spinal column from the last thoracic vertebra (T12), lumbar vertebrae (L1-L5), to the first sacral vertebra (S1). Each vertebrae is separated by a disc (visually represented as a circle) seated between the two vertebral body endplates. Two key ligaments run along the front and back of the vertebral body, the anterior and posterior longitudinal ligaments. The anterior longitudinal ligament limits extension, forward movement, and twisting of the lumbar spine. In contrast, the posterior longitudinal ligament counteracts bending of the lumbar spine. Segmental ligaments include the ligamentum flavum, and the supraspinous and interspinous ligaments. The supraspinous and interspinous ligaments are positioned between the spinous processes and serve to restrict bending of the lumbar region. The motion of the lumbar spine 30 may be characterized in three modes of loading: flexion-extension (FE), lateral bending (LB), and axial rotation (AR).

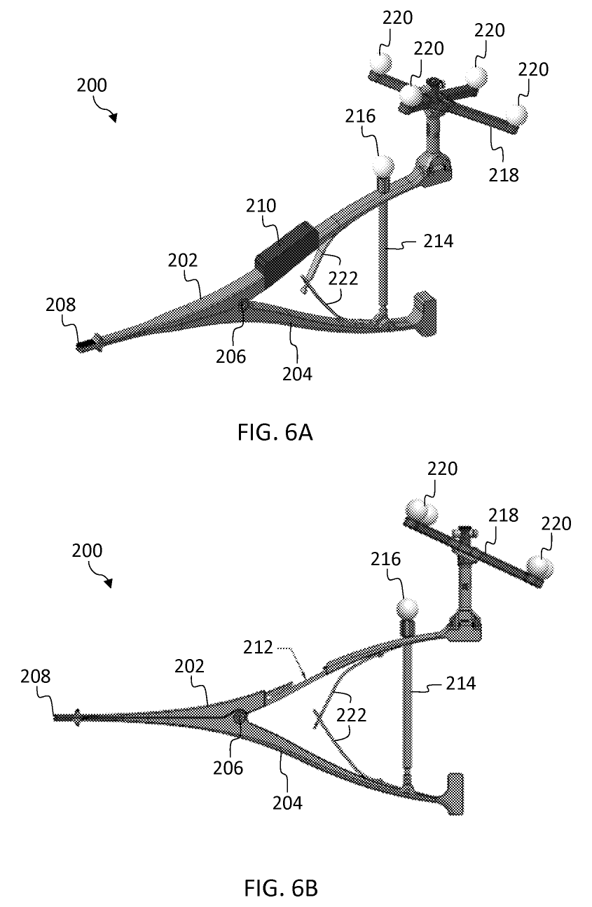

Each spinal level (e.g., T12-L1, L1-L2, L2-L3, L3-L4, L4-L5, L5-S1) may have a segmental stiffness (S₁, S₂, S₃, S₄, S₅, S₆). Segmental stiffness (SS) may be measured by applying forces F1 and F2 to adjacent vertebrae (e.g., L2 and L3). FIGS. 6A-6B depict one example of a force-sensing spreader 200 configured for measuring segmental stiffness between two vertebrae. Forces F1, F2 may include rotating the vertebrae in opposite directions and/or applying a shear load on one or more vertebrae. For example, movement of vertebra L2 may be designated by θ degrees of rotation. This rotation may cause rotation of the adjacent vertebrae (e.g., L1, L4) as well. The force F1, F2 is resisted by deformation and stiffness (S3) of the intervertebral joint (L2-L3) and also by the deformation and stiffness (S2, S4) of the adjacent joints (L1-L2 and L3-L4). Rotational stiffness may be defined as the amount of torque required to rotate one vertebra relative to another, for example, by one degree about the axis of interest. Shear stiffness may be defined as the amount of force per mm required to displace one vertebra relative to another in the transverse plane of the disc space. Segmental stiffness scores may be a measurement of the force (N) of an applied mass divided by the displacement (mm). As previously noted, these scores may be averaged or normalized for given spinal level(s), for example, for an intact healthy spine, a spine with a given deformity (e.g., scoliosis), or a spine having undergone a correction (e.g., osteotomy). The scores may also be averaged or normalized across a motion segment of multiple vertebrae. For example, a mean lumbar stiffness (MLS) may include a mean of all segmental stiffness (SS) scores across the lumbar spine 30. Global stiffness may be calculated as the slope of force-displacement (N/mm) over a given range (e.g., across the entire spine or a portion thereof). It will be appreciated that any suitable methods or calculations may be used to determine these or other spinal stiffness or range of motion values. Although only vertebrae T12-S1 are depicted, it will be appreciated that these stiffness values or other relevant data may also be obtained for the cervical and thoracic spine, or any other suitable joints.

In one embodiment, the operation may include a decompression, such as a lumbar decompression, which is used to relieve pain, for example, caused by nerve root compression. Decompression may include a microdiscectomy or discectomy, foraminotomy, laminotomy, laminectomy, laminoplasty, facetectomy, osteotomy, ligament release, foraminotomy, corpectomy, annuloplasty, interspinous spacer, interbody spacer, a combination of these, or other suitable procedures. During the procedure, the surgeon may apply forces to the spine in order to achieve the correction. The amount of force needed to correct the spinal deformity may vary from patient to patient and may be dependent upon spine stiffness. For example, a patient with a high spine stiffness may require more corrective force than a patient with a low spine stiffness. Spine stiffness may also be modified during surgery, for example, by performing osteotomies or ligament releases. If the decompression may lead to iatrogenic instability, a spinal fusion may also be performed. The fusion may include posterior fusion, for example, implementing rods and pedicle screws and/or interbody fusion by placing an implant such as a cage or bone graft within the intervertebral space following the discectomy (removing the intervertebral disc).

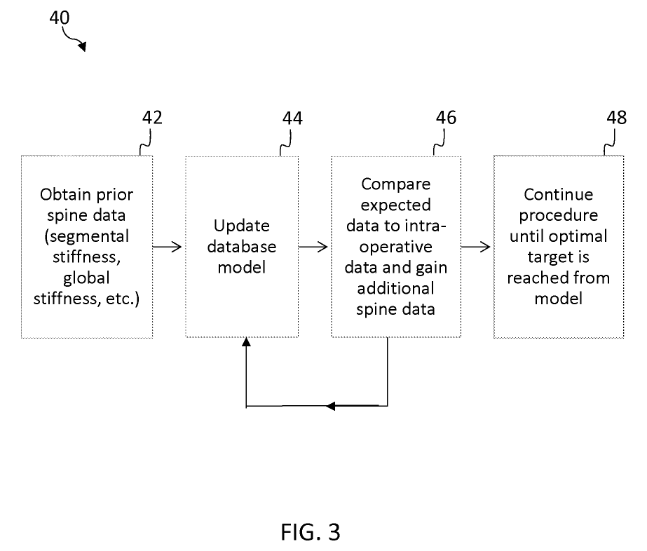

Turning now to FIG. 3, a flowchart 40 for updating the database model 10 with data collected from a patient, in real time, during an operation is shown according to one embodiment. As previously described, known spinal data (e.g., segmental stiffness, global stiffness) may be obtained in a first step 42. The database model 10 may be updated in a second step 44. The database 10 may be updated at any suitable frequency and interval to ensure the database's value, relevance, and integrity over time. During a surgical procedure, such as a lumbar decompression, the surgeon may obtain intra-operative data on the patient. For example, the surgeon may measure the actual segmental stiffness of a first spinal segment of the patient. The measured stiffness value may be compared to expected or desired stiffness values from the database 10 in a third step 46. As the spine stiffness data is collected from the patient during the procedure, the database model 10 may be iteratively updated in the second step 44. For example, once the actual segmental stiffness of the first spinal segment matches or correlates with the database model 10, the surgeon may measure the actual segmental stiffness of a second spinal segment. Again, the measured stiffness value may be compared to expected or desired values from the database 10 in the third step 46. As the spine stiffness data is collected from the patient during the procedure, the database model 10 may be iteratively updated again in the second step 44. This feedback loop may continue sequentially, in real time, to perform the decompression, level by level, until the optimal segmental stiffness is reached for each level. In other words, the decompression continues until the optimal target(s) (e.g., targeted segmental stiffness, targeted global stiffness) are reached for the patient based on the model 10 in step 48. It will be appreciated that the optimal targets may include a range or standard deviation based on the model 10. It will further be appreciated that in some cases, the model values may not be attainable for a given patient, and the procedure will continue based on surgeon judgment and expertise.

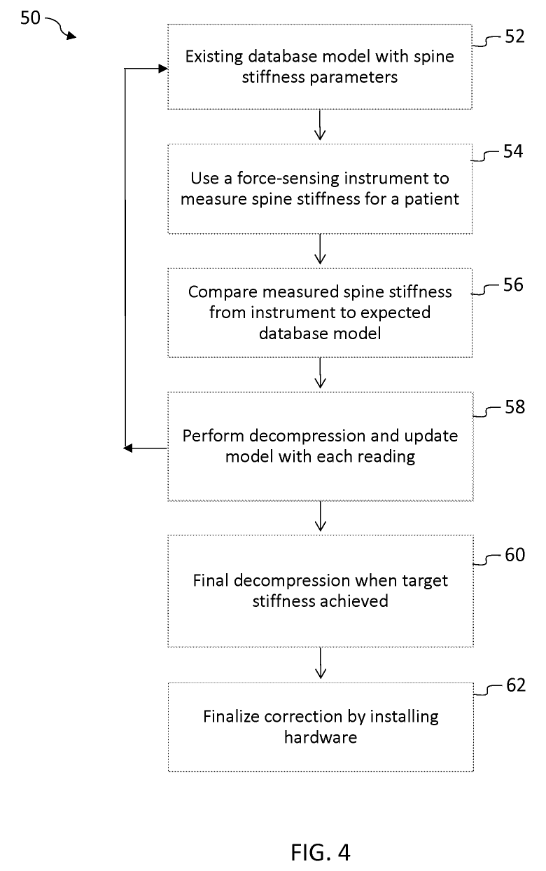

Turning now to FIG. 4, a flowchart 50 is shown for performing a spinal decompression surgery on a patient based on the database model 10. As shown in step 52, the pre-existing database model 10 includes expected and ideal spine stiffness parameters. For example, the database model 10 may include expected stiffness values for patients with a given condition (e.g., scoliosis, spondylolisthesis). The model 10 may include expected stiffness values for patients who previously underwent a decompression procedure (e.g., osteotomies or ligament releases). The model 10 may also include expected stiffness values for a patient with an intact spine or ideal outcome. These stiffness values may be represented as ranges, bar graphs, histograms, box plots, line graphs, heat maps, or other visualization methods.

As shown in step 54, during the procedure, the surgeon may use a force-sensing instrument to measure the actual spine stiffness for the patient. In step 56, the actual measured spine stiffness from the instrument is compared to the expected values from the database model 10. Based on this information, the model and/or the surgeon may be able to estimate how much movement or correction may be achieved, for example, for a given level. In some cases, the system may guide the surgeon to perform certain surgical tasks (e.g., osteotomies or ligament releases) based on the database model 10 in order to achieve the range or value of stiffness desired. This process may be iterative based on the readings obtained, the modeled parameters, and the results from the decompression techniques.

As shown in step 58, the decompression may be performed and the database model 10 may be updated with each reading. In other words, the stiffness values may include measured values before, during, and after performing a surgical task (e.g., osteotomies or ligament releases). These measured values may be added to the database model 10 to update the spine stiffness parameters in real time. The database 10 may be updated to include this new information based on a repeated feedback loop. In one embodiment, the model updates and feedback loops may be optimized, for example, using artificial intelligence (AI) and/or machine learning (ML) models. In one embodiment, the model updates and feedback may include a Baysean statistical model, which use Bayes' theorem to compute and update probabilities after obtaining new data. It will be appreciated that any suitable algorithms or models may be used to update and optimize the stiffness values or other parameters in the database 10.

As shown in step 60, this feedback loops continues until final target stiffness value(s) are achieved. In other words, the surgeon achieves final decompression when the measured values meet certain values or ranges for the expected, modeled, or ideal spine stiffness parameters. In step 62, the correction may be finalized by installing hardware, for example, to accomplish a spinal fixation and/or fusion. The fusion may include posterior fusion, for example, implementing rods and pedicle screws, interbody fusion by placing an implant such as a cage or bone graft within the intervertebral space, or any other suitable techniques.

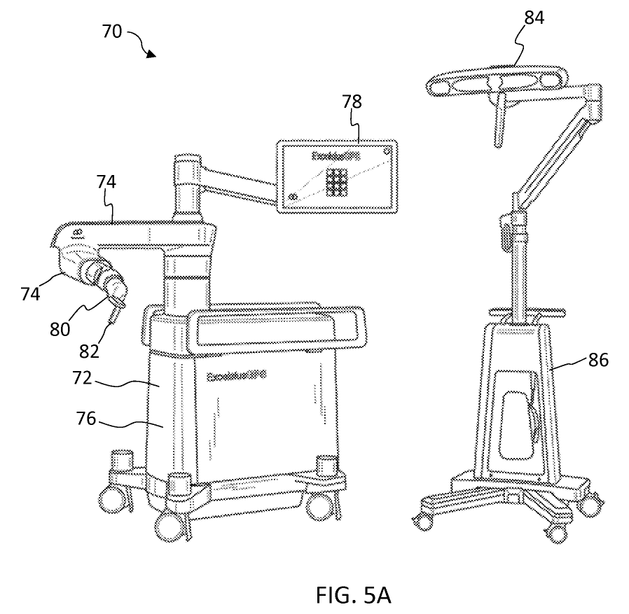

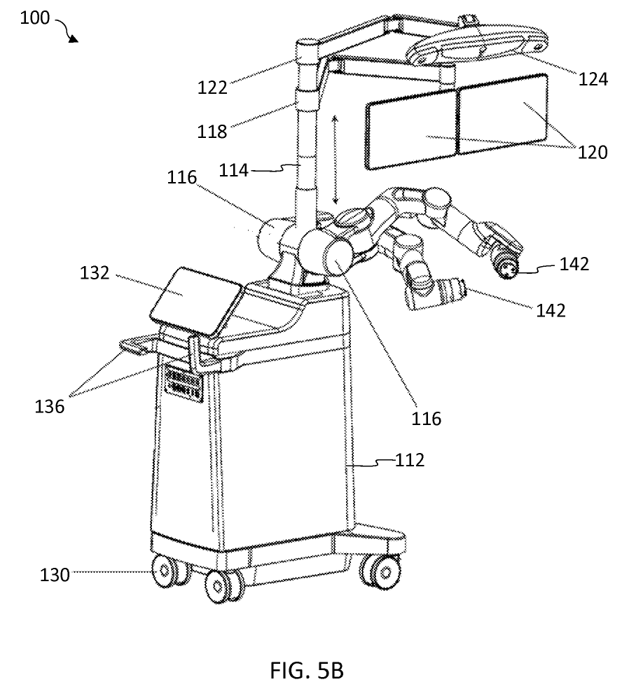

The spinal stiffness systems and methods may be incorporated into computer-assisted technology platforms, such as robotic and/or navigation systems. Surgical robotic systems with integrated navigation may include one or more surgical arms configured to assist a user with one or more surgical tasks. End effectors may be attached to each surgical arm to engage instrumentation and perform aspects of the desired surgery. Examples of surgical robotic navigation systems are shown in FIGS. 5A-5B.

FIG. 5A illustrates one example of a surgical robotic navigation system 70. The surgical robot system 70 may include, for example, a surgical robot 72, one or more robot arms 74, a moveable base 76 with one or more computers having a processor, programming, and memory, a display or monitor 78 (or optional wireless tablet) electronically coupled to the computer, and an end-effector 80, for example, including a guide tube 82 electronically coupled to the computer and movable based on commands processed by the computer. The surgical robot system 70 may also utilize a camera 84, for example, positioned on a camera stand 86. The camera stand 86 can have any suitable configuration to move, orient, and support the camera 84 in a desired position. The camera 84 may include any suitable camera or cameras, such as one or more infrared cameras (e.g., bifocal or stereophotogrammetric cameras), able to identify, for example, active and passive tracking markers in a given measurement volume viewable from the perspective of the camera 84. The camera 84 may scan the given measurement volume and detect the light that comes from the markers in order to identify and determine the position of the markers in three dimensions. For example, passive markers may include retro-reflective markers that reflect infrared light (e.g., they reflect incoming IR radiation into the direction of the incoming light), for example, emitted by illuminators on the camera 84 or another suitable device.

The robotic system 72 may include one or more computer controlled robotic arms 74 to assist surgeons in planning the position of stereotaxic instruments relative to intraoperative patient images. The system 70 includes 2D & 3D imaging software that allows for preoperative planning, navigation, and guidance through a dynamic reference base, navigated instruments and positioning camera for the placement of spine, orthopedic, or other devices. Further examples of surgical robotic and/or navigation systems can be found, for example, in U.S. Patent Publication No. 2019/0021795 and U.S. Patent Publication No. 2017/0239007, which are incorporated by reference herein in their entireties for all purposes.

FIG. 5B illustrates another example of a surgical robotic and navigation system 100. Surgical robotic system 100 may include, for example, a moveable robotic base station 112 on wheels 130, an arm positioner 114 attached to the base station 112, and multiple arms 116, 118, 122 attached to the positioner 114. Two or more surgical arms 116 may help to guide instruments or perform surgical tasks, for example, using an end effector attachable to end effector interface 142 at the distal end of each arm 116. A monitor arm 118 is configured for supporting one or more displays or monitors 120 (e.g., a dual display). A camera arm 122 is configured for supporting one or more navigation cameras 124 and/or machine vision cameras. The base 112 may support a cabinet-mounted display or terminal 132 and includes handles 136 for transporting and positioning the system 100.

In both robotic systems 70, 100, the base station 76, 112 houses an on-board computer or computing unit for controlling all functionality of the robotic system 70, 100. The on-board computer may include a central processing unit (CPU), memory, and an input/output interface. The central processing unit carries out the instructions of a computer program or software by performing arithmetical, logical, control, and input/output (I/O) operations specified by the instructions. The memory may include volatile and non-volatile memory storage that temporarily or permanently store data and instructions that are currently in use or will be needed by the central processing unit. This may include, for example, random access memory (RAM), read-only memory (ROM), and storage devices like hard drives. It will be appreciated that tangible/non-transitory computer-readable medium comprising software code or storing instructions executable by one or more processors may be adapted, when executed on a data processing apparatus, to perform any computer method set out herein. The input/output interface allows the computer system to interact with the user, take in information, and deliver results, and may include devices such as a monitor, keyboard, mouse, network interface for internet connectivity, and so forth. Although an on-board computer is exemplified herein, it will be appreciated that the computer or one or more functions may be replaced or supplemented with external devices or systems (e.g., cloud computing).

In one embodiment, the on-board computer includes the database model 10. The on-board computer may allow the robotic system 70, 100 to access and reference the spine stiffness data in real time or any other relevant information from the database model 10. The robotic system 70, 100 may process patient-specific data (e.g., actual spine stiffness readings from the patient) against the database 10 to make recommendations to the surgeon during the procedure. The robotic system 70, 100 may use the database 10 to guide the procedure (e.g., which correction maneuvers may be performed, what amount of correction can be expected). The robotic surgical arms 74, 116 may complete or assist the surgeon with the surgical task(s), the surgeon may perform the tasks with navigation assistance, or the surgeon may perform the tasks unassisted. In an exemplary embodiment, the robotic system 70, 100 may use the database 10 to identify how and where osteotomies are needed, for example, including the number and size of osteotomies. Further, the robotic system 70, 100 may identify ligament releases to loosen the spine enough to achieve the desired correction. It will be appreciated that any suitable surgical tasks, such as laminectomy, laminotomy, facetectomy, discectomy, foraminotomy, or other procedures may be used to achieve the desired spine stiffness values or other surgical outcomes. With AI or machine learning algorithms, the robotic system 70, 100 may learn from each case, contributing to the continuous update and expansion of the database 10, thereby improving its capabilities over time. With the inclusion of the spine stiffness database model 10, the robotic system 70, 100 is configured to offer real-time spine stiffness data and guidance to the surgeon, improving accuracy and overall patient outcomes.

In one embodiment, navigated force-sensing instruments may be used to characterize the stiffness of the spine and may be tracked by the robotic navigation system 70, 100. For example, the navigated force-sensing instruments may be tracked during spinal procedures when applying forces to measure and/or correct the spine. The navigated force-sensing instruments may intraoperatively measure spine stiffness on individual spine segments by measuring both displacement and force. The measured spine stiffness data may be uploaded to the database model 10 and analyzed by the robotic system 70, 100, in real time, during the surgical procedure.

The navigated instruments includes one or more markers, which are viewable and trackable by the navigation and/or robotic platform 70, 100. Infrared signal based position recognition systems may use passive and/or active sensors or markers for tracking the objects. In passive sensors or markers, objects to be tracked may include passive sensors, such as reflective spherical balls or discs, which are positioned at strategic locations on the object to be tracked. Infrared transmitters transmit a signal, and the reflective marker reflect the signal to aid in determining the position of the object in 3D. In active sensors or markers, the objects to be tracked include active infrared transmitters, such as light emitting diodes (LEDs), and generate their own infrared signals for 3D detection.

In one embodiment, the trackable markers may include radiopaque or optical markers. The markers may be suitably shaped, including spherical, spheroid, disc, cylindrical, cube, cuboid, or the like. The trackable markers may be coupled to the surgical instrument in any appropriate manner. The trackable markers may include fixed or movable markers used to measure forces to or on the instrument or due to forces of or applied to the associated anatomy. Alternatively, machine vision may be employed to track the instruments without any markers.

The navigated force-sensing instruments may include any suitable instruments used for applying forces to move the spine before or during correction, for example, in compression, distraction, reduction, and/or derotation. The instruments may include a compressor configured to compress vertebrae, including parallel or angled compression, a distractor configured to distract vertebrae, including parallel or angled distraction, a reducer configured to provide movement to translate and/or derotate the spine, and/or a rib pusher configured to apply force to the ribs. Although certain instruments are exemplified herein, it will be appreciated that the force-sensing instrument may include any instrumentation utilized in spinal fusion procedures or other surgical procedures.

Turning now to FIGS. 6A-6B, a navigated force-sensing instrument 200 is shown according to one embodiment. The navigated force-sensing instrument 200 may include a spreader instrument with built-in force measuring, wireless communication, and navigation tracking. The instrument 200 may include two pivotable arms 202, 204 connected by a hinge 206 with distal tips 208 configured to engage the spine, an electronics package 210 around a sensing portion 212 on one arm 202, 204, a ratchet 214 with a reflective marker 216, and a navigation array 218 with reflective markers 220 for instrument tracking.

The force-sensing spreader instrument 200 may have a first arm 202 and an opposed second arm 204 configured to engage bone. The first and second arms 202, 204 may be interconnected at hinge or pivot pin 206. The distal tips 208 of the instrument 200 may include tabs, prongs, or suitable geometry configured to engage a specific area of the spine, such as the spinous process, lamina, or vertebral body. The proximal ends of each arm 202, 204 are manipulatable by a user, such as a surgeon. For example, the first and second arms 202, 204 may each define a handle toward the proximal end, which are configured to be gripped and squeezed by the user. The inner facing portions of the handles may include curved leaf springs 222 configured to keep the distal tips 208 of the instrument 200 closed at rest.

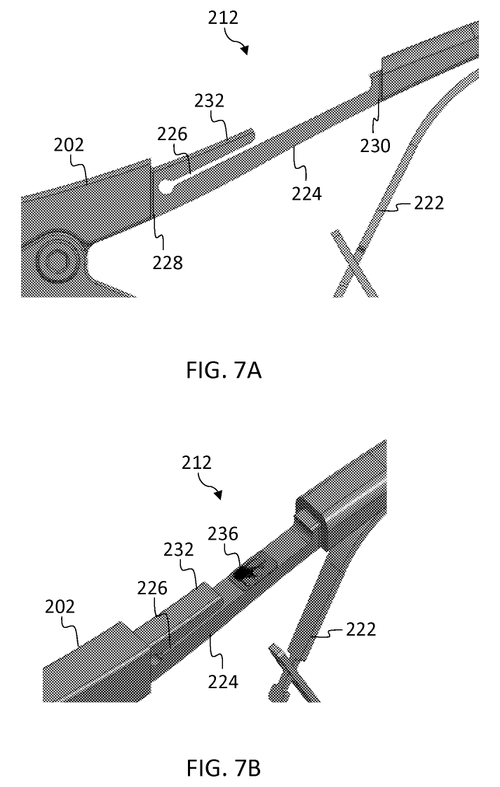

As best seen in FIG. 7A, one arm 202, 204 of the instrument 200 includes a sensing portion 212. The arm 202, 204 may include a cutout 224 connected with a wire cut 226 along the long axis of the instrument arm 202, 204 to create the sensing portion 212 of the instrument 200. The cutout portion 224 may have a decreased width, depth, thickness, or diameter compared to the rest of the arm 202, 204. The cutout portion 224 may narrow in width or thickness from a distal end 228 toward a proximal end 230 of the cutout 224. One side of the cutout 224 may include a relief cut or wire cut 226. The wire cut 226 may include a circular or semi-circular cut toward the distal end 228 of the cutout portion 224, which is in fluid communication with a slit extending toward the proximal end 230 of the cutout 224. The wire cut 226 forms a free tab 232 having a free end facing toward the proximal end of the arm 202, 204. The sensing portion 212 forms a strain bridge with reduced cross-section, which allows it to flex more than other areas of the instrument 200 during use.

As best seen in FIG. 7B, one or more strain gages 236 may be secured to the strain bridge 212 in order to measure the strain experienced by the instrument 200. The strain gage 236 may include a sensor or transducer configured to measure strain or deformation of the cutout 224. When the arm 202, 204 is subjected to stress or force, the cutout 224 may deform or flex, and the strain gage 236 can detect and measure the deformation or flexure. The strain gage 236 may be connected to the electronics package 210 positioned around the sensing portion 212 of the instrument 200.

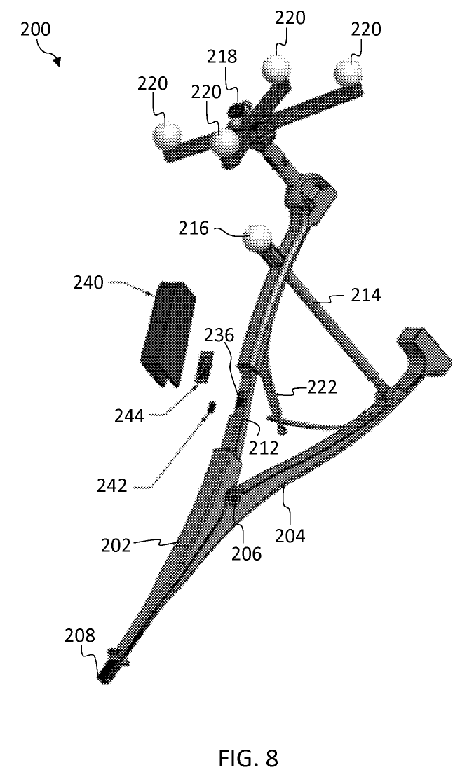

As best seen in FIG. 8, the electronics package 210 may include a housing 240, a battery 242, and a circuit board 244 with a wireless communication module. The circuit board 244 may receive and filter signals from the strain gage(s) 236. The communication module may be located on the circuit board 244 and sends the signals wirelessly to an external computer, such as the on-board computer for the robotic system 70, 100. The wireless communication may function through radio frequency (RF) modalities such as Zigbee, Bluetooth, Bluetooth LE, or Wi-Fi. The battery 242 provides power to the electronics in the device and may be retained or removeable from the instrument 200. The electronics package 210 may also include an induction coil, which allows the battery 242 to be recharged wirelessly. Alternatively, the battery 242 may be replaced with a new one after each use. The electronic components 242, 244 are secured within the housing 240 and positioned around the strain bridge 212 in close proximity to the strain gages 236. The electronics housing 240 may be made of a non-ferrous material, such as polyether ether ketone (PEEK), acrylonitrile butadiene styrene (ABS), polylactic acid (PLA), or other plastic material that allows RF communication. The electronics package 210 may also have a light-emitting diode (LED) or other indicator that lights up to indicate information to the user. For example, the LED may signify the instrument 200 is on or off. Alternatively, the indicator may indicate when the instrument 200 is currently measuring force. Furthermore, the LED may indicate the amount of force by changing color based on the amount of force detected.

The instrument 200 may include a ratchet 214 configured to hold the relative position of the arms 202, 204 to allow for precise, incremental adjustments, and secure locking of the instrument's position during use. In one embodiment, ratchet arm 214 may be positioned between the handle portions of the first and second arms 202, 204. The ratchet arm 214 may be affixed to the second arm 204 and positioned through a slot or opening 234 in the first arm 202 or vice versa. For example, the ratchet arm 214 may include a cylindrical body with teeth, steps, or threads configured to mate with a corresponding tooth or pawl in the opening 234. The ratchet mechanism may allow for movement in one direction while preventing movement in the opposite direction until intentionally released. When squeezed, the ratchet arm 214 is configured to incrementally maintain the relative positions of the distal tips 208 of the arms 202, 204 and the amount of force applied to the vertebrae.

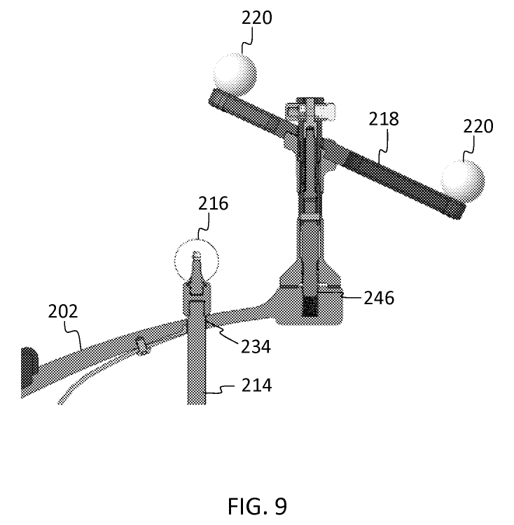

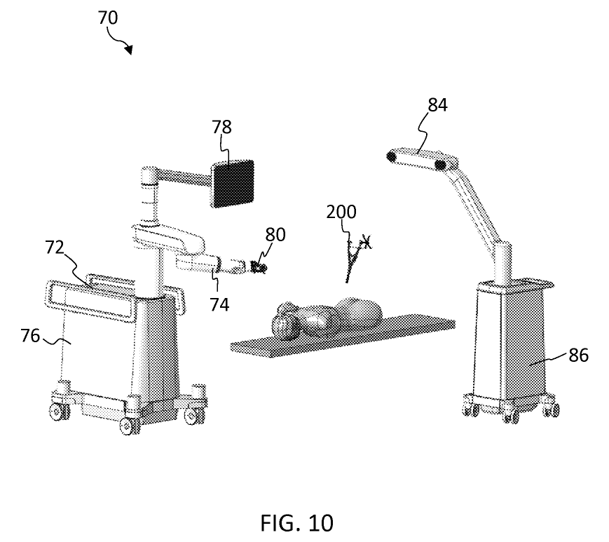

The navigation array 218 may be permanently affixed to the instrument 200 or the array 218 may be reversibly attachable. The array 218 may include a post attached to the proximal-most end of one arm 202, 204. A unique array pattern may attach to the post to identify and differentiate the instrument 200 from other objects being navigated. The tracking markers 220 may be affixed to the array 218, for example, four tracking markers 220 in the pattern shown. As shown in FIG. 9, the array 218 may be detachable and connected to the arm 202 with a threaded connection 246. In this embodiment, the array 218 may have reflective markers 220 or active infrared LEDs that can be tracked by the navigation camera located in the operating room. For example, the instrument 200 may be compatible with the cameras 84, 124 of robotic and navigation systems 70, 100 or other suitable navigation techniques. FIG. 10 depicts an operating room setup with a patient on the operating room table. The force-sensing spreader 200 may be manipulated by the surgeon while the force-sensing spreader 200 is tracked by robotic navigation system 70 in real time. By continuously monitoring the positions of the markers 220 on the array 218, the system 70 can monitor and track the precise location, position, and orientation of the instrument 200 throughout the surgery.

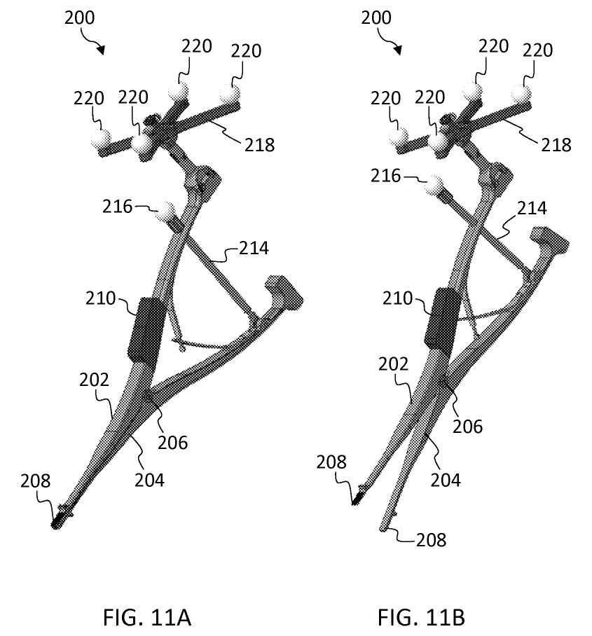

The spreader instrument 200 also has a single reflective marker 216 or active infrared LED on the ratchet arm 214. The ratchet marker 216 may be located at the free end of the ratchet arm 214 on the outside of the pivoting arm 202. As shown in FIGS. 11A-11B, the spreader 200 has a closed condition (left) and an open condition (right). When at rest, as shown in FIG. 11A, the ratchet marker 216 is positioned adjacent to arm 202. When the instrument 200 is squeezed, as shown in FIG. 11B, the ratchet arm 214 protrudes from arm 202 and extends ratchet marker 216 outward and closer to array 218. The movement of ratchet marker 216 allows the system 70, 100 to track the distance that the distal tips 208 are opened or spread apart. The difference in distance between the ratchet marker 216 and the array 218 may be used by system 70, 110 to ascertain the movement of distal tips 208. In particular, the distance may be determined by calculating the relationship between the current position of the ratchet marker 216 and the current position of the array markers 220 since the distance between them changes depending on how far the distal tips 208 are opened.

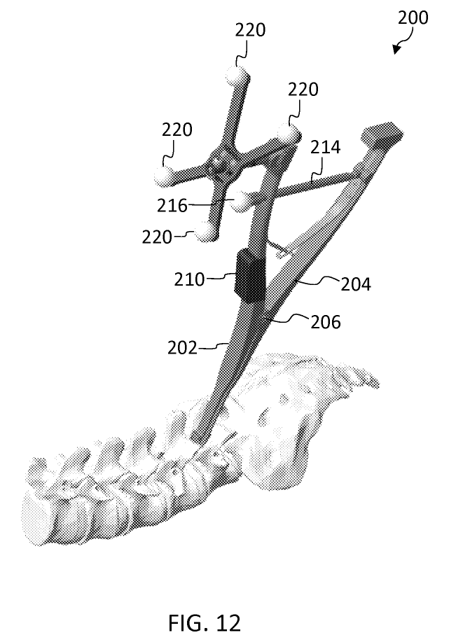

Turning now to FIG. 12, the force-sensing spreader 200 is placed between two boney structures on adjacent vertebrae. The user squeezes the handle arms to open the distal tips 208, thus applying force to displace the adjacent vertebrae away from one another. The force exerted on the instrument 200 causes the sensing portion 212 of the arm 202 to experience strain, which is detected by the strain gage 236 placed on the strain bridge 212. The signals generated by the strain gage 236 are communicated to the external computer via the communication module on the circuit board 244.

Stiffness may be calculated as the applied force divided by the deflection. Software on the external computer receives the strain data from the instrument 200 to calculate the applied force and position data from the camera 84, 124 to calculate the deflection. Therefore, the system 70, 100 is able to calculate segmental spine stiffness at the level where the instrument 200 is used. The surgeon may move between different levels of the spine and use the instrument 200 to measure segmental spine stiffness at each level. Since the system 70, 100 is able to track the position of the instrument(s) 200, the system 70, 100 may automatically calculate and record spine stiffness at each level without any prompts from the user.

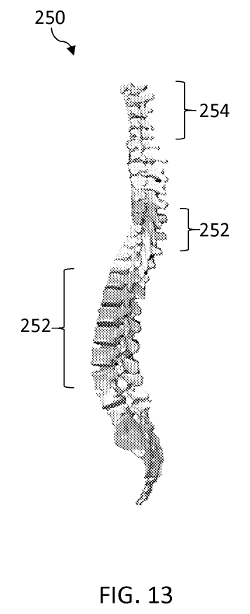

As shown in FIG. 13, a schematic or virtual representation 250 of the spine may be shown to the user on the monitor(s) 78, 120 during the procedure, which indicates the spine stiffness at each level or trends along motion segments. The schematic representation 250 may show spine stiffness, for example, by showing a color gradient representing the spine stiffness between levels (e.g., red representing a higher stiffness, green representing a lower stiffness, and orange and yellow transitioning between red and green areas). Red or darker areas 252 may denote higher stiffness values, and green or lighter areas 254 may denote lower stiffness values. It will be appreciated that the visual representation or gradient would be unique to each individual patient depending on the measured values or anticipated values, for example, from the database model 10. The spine stiffness measurements may be displayed on the monitor 78, 120 to the user in various formats, such as actual measurements, graphical depictions of relative stiffness for each spine level, or comparative analysis against normative data benchmarks, enabling a comprehensive and intuitive understanding of the patient’s spinal condition.

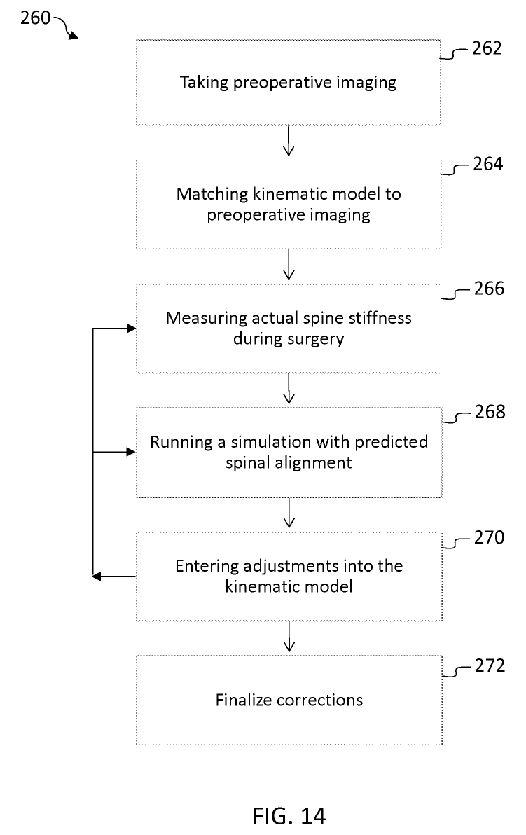

Turning now to FIG. 14, a simulation method 260 for simulating deformity correction using a kinematic model of the spine is shown according to one embodiment. In a first step 262, preoperative imaging, such as fluoroscopy, computed tomography (CT), or magnetic resonance imaging (MRI) may be taken of the patient to determine an initial alignment of the spine. In a second step 264, the initial shape of the kinematic model may be configured to match the initial alignment of the patient’s spine determined from the preoperative imaging. Behavior conditions of the kinematic model may be configured based on patient specific information, such as age, weight, sex, bone mineral density, and spine stiffness. This information may be obtained from patient health records, preoperative measurements, physical exams, or other medical assessments. The kinematic model may define a relationship between each vertebra such that it can estimate how each vertebra moves in response to a given force or moment input. Spine stiffness influences the kinematics of the spine. High spine stiffness causes a decrease in movement for a given force input, and conversely, low spine stiffness causes an increase in movement for a given force input.

In a third step 266, during surgery, the spine is exposed and the force-sensing spreader 200 or other instrument may be used to measure the spine stiffness at each level of interest. The stiffness measurement for each level may be recorded and assigned to the respective level in the kinematic model. Force and moment data may be input based on the surgeon’s chosen correction technique. In a fourth step 268, a simulation is run with the predicted spine alignment resulting from the chosen correction technique is displayed to the surgeon. The surgeon analyzes the predicted deformity correction from the kinematic model and decides whether to adjust the surgical plan. Adjustments to the surgical plan may include additional osteotomies, more aggressive osteotomies, different correction techniques, or different instrumentation.

In a fifth step 270, adjustments are input into the kinematic model and another simulation (step 268) may be run again to create an updated deformity correction prediction. If additional interventions were performed to change the stiffness of the patient’s spine, then the force-sensing spreader 200 may be used again (step 266) to measure the new spine stiffness at each level. The new stiffness measurements are then updated in the kinematic model before a new simulation (step 268) is performed. This feedback loop of measurement and prediction may be repeated until the desired deformity correction is achieved for that patient. In a final step 272, the surgeon completes the corrections and installs hardware to finalize the correction. This workflow may be useful in allowing the surgeon to optimize the strategies for changing the patient’s spine stiffness to achieve correction by balancing correction forces and anatomical disruption.

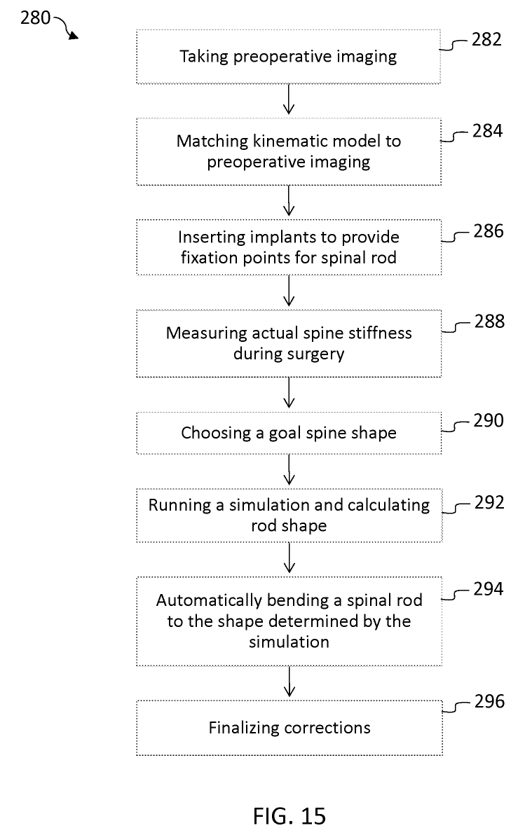

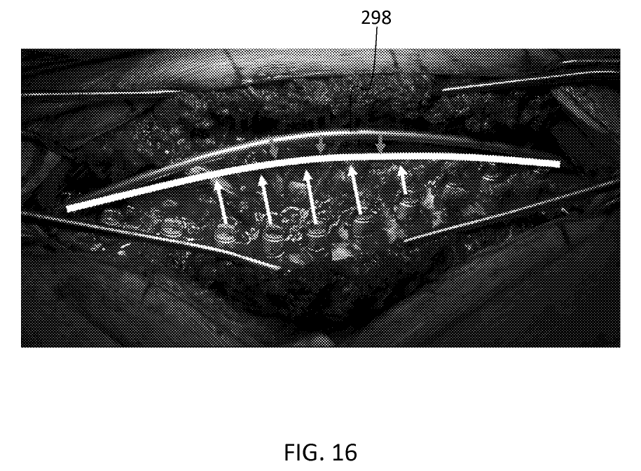

Turning now to FIG. 15, a method 280 for creating a patient specific spinal rod intraoperatively using spine stiffness measurements and a kinematic model of the spine is shown according to one embodiment. As shown in FIG. 16, spinal rods 298 are used during spine deformity surgery to achieve correction and to hold the correction postoperatively. The patient’s spine stiffness influences the forces acting on the rod 298. High spine stiffness places more force on the rod 298 during correction while low spine stiffness places less force on the rod 298 during correction. This phenomenon influences surgical decision-making regarding rod material and diameter. Large stiff deformities may require larger stiffer rods. The forces acting on the rod from the spine oftentimes deform the shape of the rod during correction. As a result, surgeons may overbend the rod, knowing that it will flatten out during correction, in order to end up with the desired rod shape post-correction. Since spine stiffness influences the amount of force acting on the rod 298 and therefore what the shape of the rod should be before correction maneuvers are performed, measuring the spine stiffness of the patient intraoperatively provides more valuable data to the surgeon.

The method 280 for creating a patient specific spinal rod intraoperatively using a kinematic model of the spine may include the following steps. In a first step 282, preoperative imaging such as fluoroscopy, CT, or MRI may be taken of the patient to determine an initial alignment of the spine. In a second step 284, the initial shape of the kinematic model may be configured to match the initial alignment of the patient’s spine determined from preoperative imaging. Behavior conditions of the kinematic model are configured based onpatient specific information. In a third step 286, during surgery, implants may be inserted into the spine to provide points for fixation to the rod. In a fourth step 288, the force-sensing spreader 200 or other instrument may be used to measure the spine stiffness at each level of interest. The stiffness measurement for each level may be recorded and assigned to the respective level in the kinematic model. Force and moment data may be input based on the surgeon’s chosen correction technique. In a fifth step 290, a goal spine shape may be chosen, which reflects what the surgeon would like the patient’s spine to look like after surgery. In a sixth step 292, a simulation may be run and a patient specific rod shape may be calculated based on a number of variables including the patient’s initial spine shape, the surgeon’s goal spine shape, rod diameter, rod material, correction technique, and/or the spine stiffness measured intraoperatively from the force-sensing spreader 200. In a seventh step 294, the patient specific rod shape may be sent to an automatic rod bender, which bends the spinal rod intraoperatively to the exact shape determined by the simulation. In a final step 296, the surgeon performs the correction using the patient specific rod and completes the surgery.

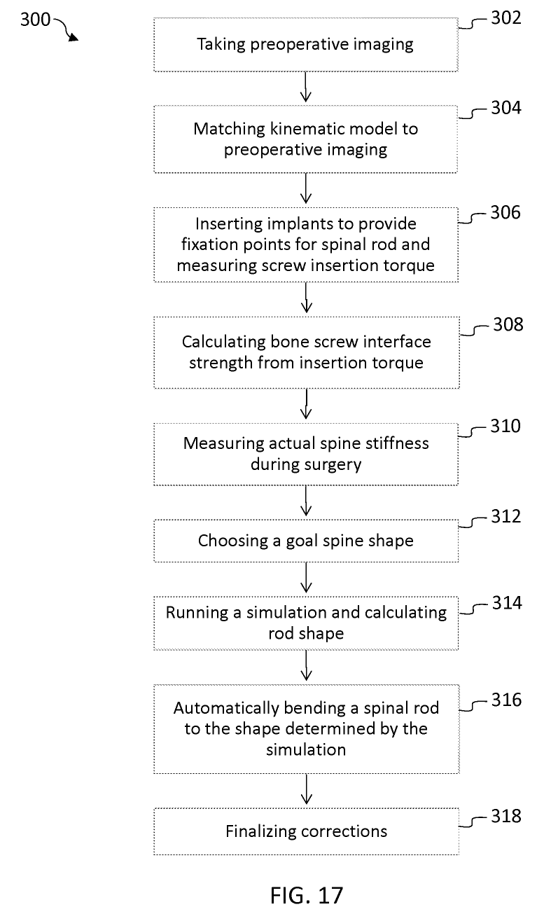

Turning now to FIG. 17, a method 300 for creating a patient specific spinal rod intraoperatively using bone screw interface strength, spine stiffness measurements, and a kinematic model of the spine is shown according to one embodiment. In this embodiment, intraoperative measurement of screw insertion torque may be used to influence the ideal rod shape calculated by the system. In order to achieve correction during spine deformity surgery, instruments may be used to push and pull the spine from the deformed shape to the corrected shape. The force required to push and pull the spine from the deformed shape to the corrected shape depends on the patient’s spine stiffness. Higher stiffness requires more force to achieve a certain amount of correction. However, there is a limit to how much force can be placed on the spine implants before the bone screw interface fails. Therefore, spine deformity correction may be a balancing act between achieving correction and minimizing stress placed on the implants. In order to know how much force can be safely placed on the implants during deformity correction, bone screw interface strength may be measured intraoperatively and added to the kinematic model. In this embodiment, a torque measuring instrument may be used to calculate the bone screw interface strength.

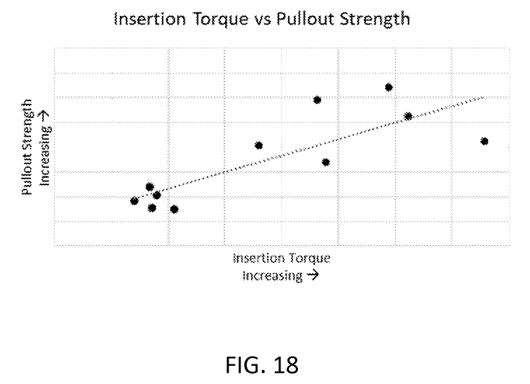

The method 300 for creating a patient specific spinal rod intraoperatively using a kinematic model of the spine may include the following steps. In a first step 302, preoperative imaging such as fluoroscopy, CT, or MRI may be taken of the patient to determine an initial alignment of the spine. In a second step 304, the initial shape of the kinematic model may be configured to match the initial alignment of the patient’s spine determined from preoperative imaging. Behavior conditions of the kinematic model are configured based on patient specific information. In a third step 306, during surgery, implants are inserted into the spine to provide points for fixation to the rod. A torque measuring instrument may be used to insert screws into the spine. The screw insertion torque may be recorded and assigned to the respective location on the spine in the kinematic model. In a fourth step 308, a calculation of bone screw interface strength may be made based on the screw insertion torque such that a pullout force is predicted for each screw placed in the spine. As shown in the chart of FIG. 18, higher screw insertion torque may result in higher predicted pullout force.

In a fifth step 310, the force-sensing spreader 200 or other instrument may be used to measure the spine stiffness at each level of interest. The stiffness measurement for each level may be recorded and assigned to the respective level in the kinematic model. Force and moment data may be input based on the surgeon’s chosen correction technique. In a sixth step 312, a goal spine shape may be chosen which reflects what the surgeon would like the patient’s spine to look like after surgery. In a seventh step 314, a simulation may be run and a patient specific rod shape may be calculated based on a number of variables including the patient’s initial spine shape, the surgeon’s goal spine shape, rod diameter, rod material, correction technique, bone screw interface strength measured intraoperatively from the torque sensing instrument, and/or spine stiffness measured intraoperatively from the force-sensing spreader 200. The simulation may choose a rod shape that ensures the force placed on each screw during correction would not exceed the predicted pullout force of that screw. In an eight step 316, the patient specific rod shape may be sent to an automatic rod bender, which bends the spinal rod intraoperatively to the exact shape determined by the simulation. In a final step 318, the surgeon then performs the correction using the patient specific rod and completes the surgery.

In another embodiment, methods 280, 300 may also include intraoperative measurement of patient bone quality to influence the ideal rod shape calculated by the system. Bone quality determines how much force can be placed on an implant before it becomes dislodged from the bone. Low bone density typically means lower bone screw interface strength, while high bone density typically means high bone screw interface strength. Since spine deformity correction requires surgeons to push and pull on implants fixated within the bone, bone quality may be considered when performing spine deformity surgery. In order to know how much force can be safely placed on the implants during deformity correction, measuring the patient’s bone quality intraoperatively further provides more valuable data to the surgeon. In this embodiment, an intraoperative bone mineral density calibration device may be used to calculate bone quality.

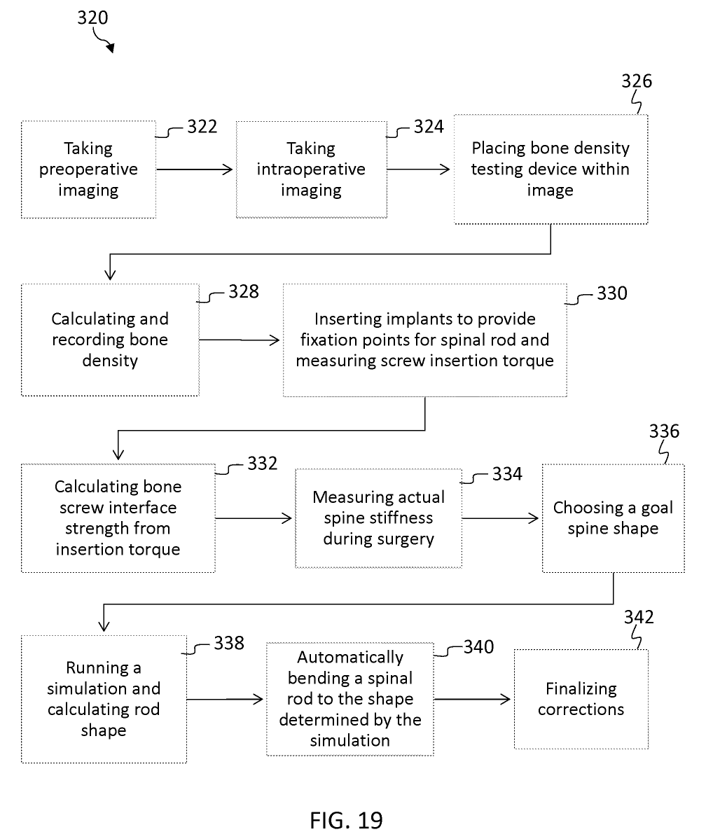

Turning now to FIG. 19, the method 320 for creating a patient specific spinal rod intraoperatively using a kinematic model of the spine may include the following steps. In a first step 322, preoperative imaging such as fluoroscopy, CT, or MRI may be taken of the patient to determine an initial alignment of the spine. The initial shape of the kinematic model may be configured to match the initial alignment of the patient’s spine determined from preoperative imaging. Behavior conditions of the kinematic model are configured based on patient specific information. In a second step 324, at the beginning of surgery, intraoperative fluoroscopy or CT may be used to identify the location of anatomy in 3D space. In a third step 326, the bone mineral density calibration device may be placed within the image. In a fourth step 328, software may be used to calculate the bone mineral density of each level of interest based on comparisons in the image of the anatomy and phantoms housed within the bone mineral density calibration device. The measurement of bone mineral density for each level may be recorded and assigned to the respective level in the kinematic model.

In a fifth step 330, implants may be inserted into the spine to provide points for fixation to the rod. A torque measuring instrument may be used to insert screws into the spine. The screw insertion torque may be recorded and assigned to the respective location on the spine in the kinematic model. In a sixth step 332, a calculation of bone screw interface strength may be made based on the measured screw insertion torque and bone mineral density such that a pullout force is predicted for each screw placed in the spine. In a seventh step 334, the force-sensing spreader 200 or other instrument may be used to measure the spine stiffness at each level of interest. The stiffness measurement for each level may be recorded and assigned to the respective level in the kinematic model. Force and moment data may be input based on the surgeon’s chosen correction technique. In the eight step 336, a goal spine shape may be chosen, which reflects what the surgeon would like the patient’s spine to look like after surgery.

In a ninth step 338, a simulation may be run and a patient specific rod shape may be calculated based on a number of variables including the patient’s initial spine shape, the surgeon’s goal spine shape, rod diameter, rod material, correction technique, bone mineral density measured intraoperatively from the calibration device, bone screw interface strength measured intraoperatively from the torque sensing instrument, and/or spine stiffness measured intraoperatively from the force-sensing spreader 200. The simulation may choose a rod shape that ensures the force placed on each screw during correction could not exceed the predicted pullout force of that screw, which was informed by the measurements of screw insertion torque and bone mineral density. In a tenth step 340, the patient specific rod shape may be sent to an automatic rod bender, which bends the spinal rod intraoperatively to the exact shape determined by the simulation. In the final step 342, the surgeon performs the correction using the patient specific rod and completes the surgery.

Accurate measurement of spine stiffness allows the surgeon to create an optimized surgical plan to correct the patient’s spinal deformity based on that individual’s unique spine stiffness. The spreader instrument provides for an accurate and uniform method of measuring spine stiffness, as opposed to techniques which may be subjective or rely on patient compliance and may lead to inaccuracies. The spreader instrument allows for a uniform method of stiffness measurement that is independent of actions performed by the patient. In addition, it provides a spine stiffness measurement at the time of surgery, thereby allowing the surgeon to decide the most appropriate level of surgical intervention and immediately executing that decision. The spine stiffness data provides the surgeon with a better understanding of the patient’s specific spinal biomechanics and may allow the surgeon to offer better and more accurate surgical correction, as well as eliminate some of the subjective judgments used during surgery.

In addition to spine stiffness measurements taken before correction, it may also be useful to obtain force measurements during the correction of the spinal deformity. The correction forces may be applied to the bones (e.g., vertebrae) or to the implants (e.g., bone screws or rods). When applying force directly to the bone screw, the amount of force which may be applied by the surgeon may also be dependent upon the bone screw interface. In other words, the strength of the bone screw interface may dictate how much force the surgeon can apply to the spine before the implant dislodges from the bone, which could cause injury and loss fixation to the patient. The strength of the bone screw interface in patients with low bone mineral density may be weaker than in patients with high bone mineral density. Therefore, surgeons may use lower corrective forces in patients with low bone mineral density, which may also limit the amount of correction that may be achieved in that patient. In order to maximize safe correction in spine deformity surgery, it may be helpful for surgeons to know how much force they are exerting on the patient during the correction. Current methods of measuring corrective forces are subjective and rely on surgeon feel and judgment. Instead, devices that measures correction force during surgery allow the surgeon to understand the true amount of force on the patient. The devices and methods of measuring spine deformity correction forces intraoperatively may decrease the risk of implant dislodgment and allow for better and safer correction of spine deformity.

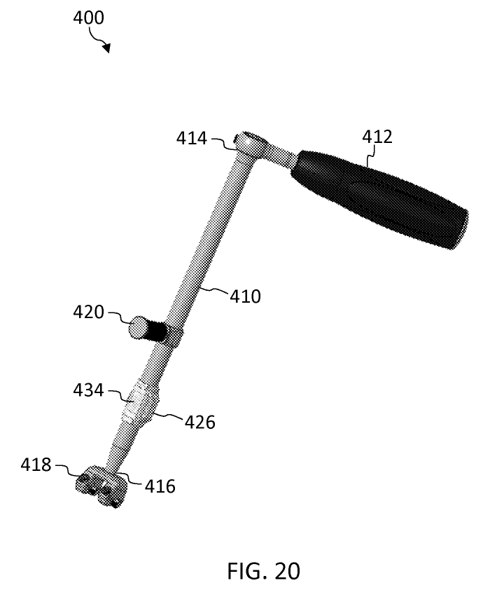

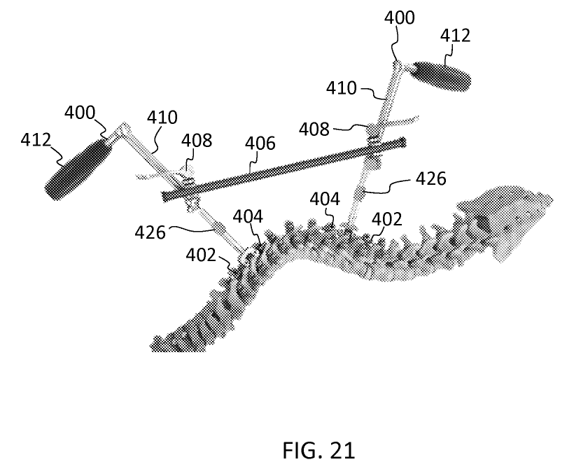

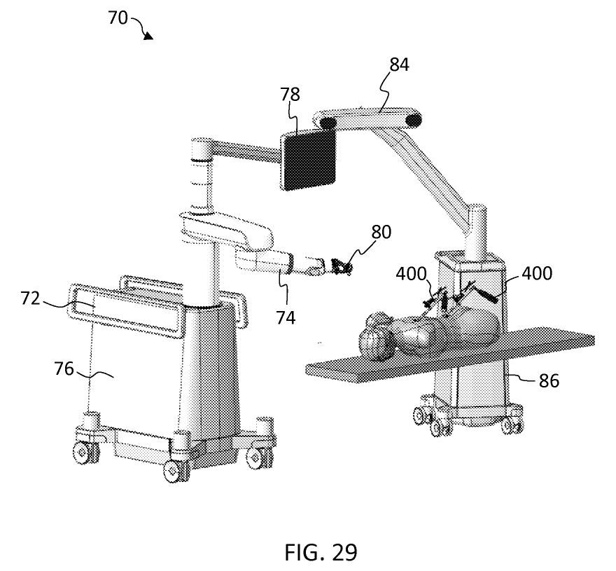

Turning now to FIGS. 20-29, a rod link reducer 400 with built-in force measuring and wireless communication is shown according to one embodiment. FIG. 21 depicts anatomy of the spine and two temporary spinal constructs each including a provisional spinal rod 402 attached to pedicle screws 404, a pair of rod link reducers 400 for manipulating the respective spinal constructs, and a cross shaft or coupling rod 406 for locking the correction accomplished by the reducers 400. Once the deformity is corrected, a permanent spinal rod may be placed on the opposite side of the spine to permanently maintain the deformity correction. Examples of rod link reducers and methods for correcting spinal deformities are described in more detail, for example, in U.S. Patent Nos. 10,869,699 and 10,736,671, which are incorporated by reference herein in their entireties for all purposes.

As best seen in FIG. 20, the rod link reducer 400 may include a manipulating arm or manipulator rod 410 and a handle 412. The manipulator rod 410 has a first end or proximal end 414, a second end or distal end 416, and an elongate body extending along a body axis between first end 414 and second end 416. The handle 412 may be coupled to the manipulating arm 410 at the proximal end 414 and configured to be held and manipulated by the surgeon. The handle 412 may be positioned generally orthogonal to the body axis of the manipulator rod 410. The handle 412 is adapted to maneuver the manipulator rod 410 as desired by the surgeon.

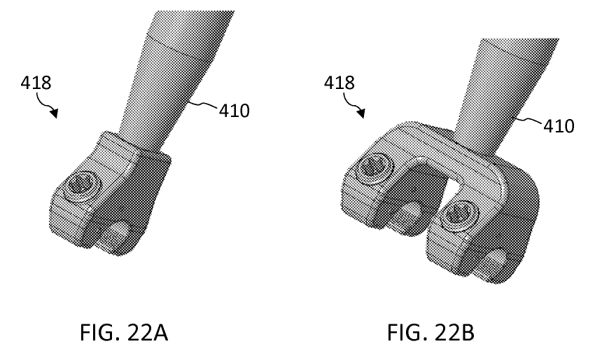

The second end 416 of manipulator rod 410 includes a distal tip or clamping portion 418 with a receiver adapted to releasably secure the temporary spinal rod construct therein. As best seen in FIGS. 22A-22B, the clamping portion 418 may include a single style (left) or double style (right) manipulating arm. The single style is configured to connect to rod 402 between two pedicle screws 404. The double style is configured to attach to rod 402 with each clamp positioned between two pedicle screws 404, one of which is positioned between the two clamps. The clamping portion 418 is sized to releasably receive and retain the spinal rod 402 therein. The spinal rod 402 may be retained within the distal receiver 418 of the rod link reducer 400 via one or more securing members, such as set screws, configured to engage and secure the spinal rod 402. It will be appreciated that the manipulator rod 410 may be temporarily secured to the temporary spinal construct in any suitable manner and manipulated to perform the desired surgical corrections.

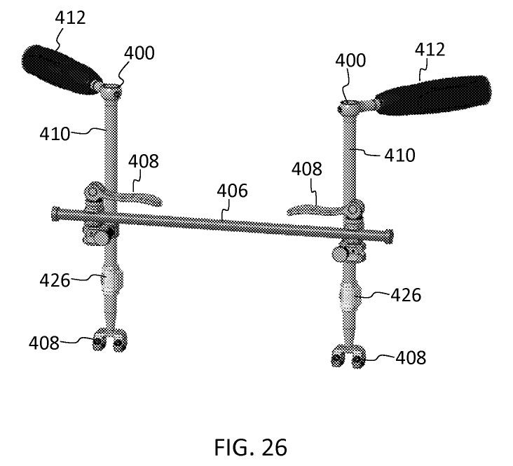

The manipulating arm 410 may include a post 420 for attaching the coupling rod 406 with a clamp 408 or other securing member. The post 420 may be oriented perpendicular to the long axis of the manipulating arm 410. The post 420 is configured to attach to the coupling clamp 408. The coupling clamp 408 may have two clamping portions, one for attachment on the post 420 and one for securing the coupling rod 406. Two rod link reducer instruments 400 may be coupled together via the coupling clamps 408 and coupling rod 406 to perform the desired correction, as shown in FIG. 26.

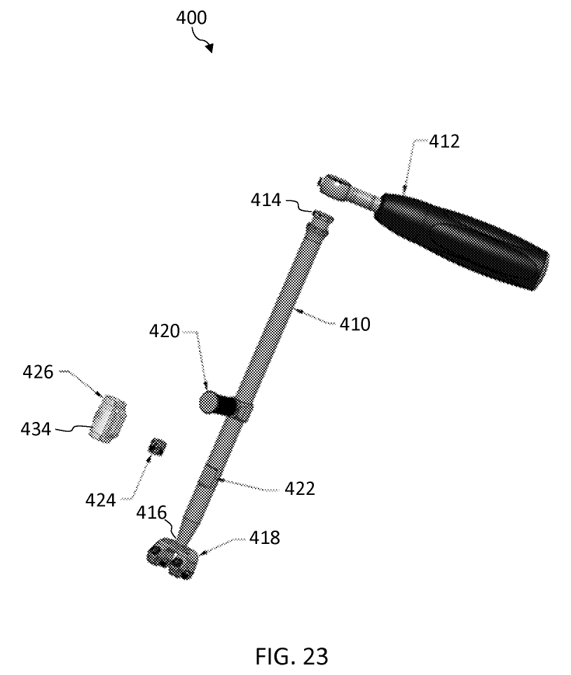

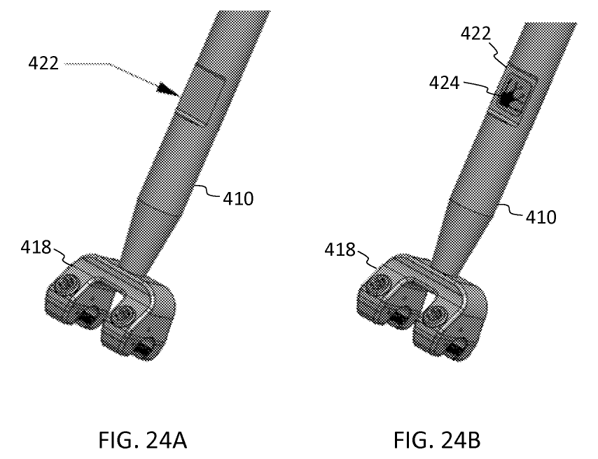

As shown in FIG. 23, the rod link reducer 400 includes a strain bridge 422, one or more strain gages 424, and an electronics package 426. Similar to instrument 200, a portion of the shaft of the manipulating arm 410 has geometry configured to form a sensing portion or strain bridge 422. The strain bridge 422 forms an area with reduced cross section, for example, having a decreased width, depth, thickness, or diameter compared to the rest of the arm 410. The reduced cross section causes that area to experience increased strain during loading. As best seen in FIGS. 24A-24B, the strain bridge 422 may define a flat surface on which the strain gages 424 can be mounted or affixed. Strain gages 424 may be secured to the strain bridge 422 in order to measure the strain experienced by the instrument 400. In one embodiment, the strain gages 424 may be configured in a strain gage rosette or other pattern in order to allow measurements of strain in multiple directions. A rosette pattern (e.g., multiple strain gages 424 positioned at predetermined angles relative to each other) allows the instrument 500 to measure force exerted onto the patient in multiple planes. The strain gages 424 may be arranged in a specific pattern (e.g., 0°, 45°, 90° arrangements or 0°, 60°, 120° arrangements) for accurate determination of a multi-axial strain state, thereby enhancing the capability to measure and analyze strain on the instrument 400.

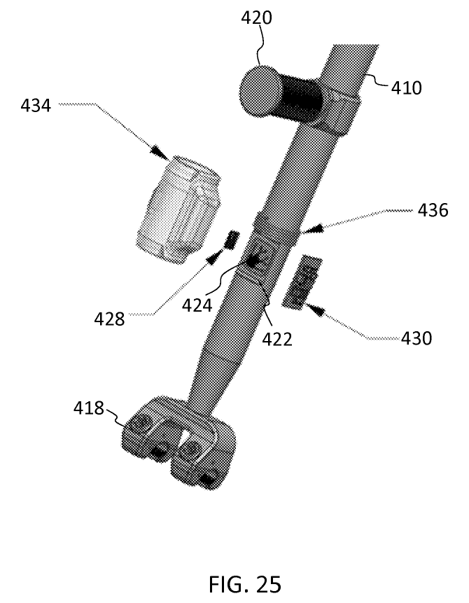

As shown in the exploded view in FIG. 25, the strain gages 424 are connected to electronics package 426. The electronics package 426 may include a battery 428, a circuit board 430 with a wireless communication module, and an outer housing 432. The circuit board 430 may receive and filter signals from the strain gages 424. The wireless communication module receives the signals and communicates them to an external computer, such as on-board computers for robotic systems 70, 100. The wireless communication may function through radio frequency (RF) modalities such as Bluetooth, Bluetooth LE, or Wi-Fi. The battery 428 powers the electronics in the instrument 400. The electronics package 426 may also include an induction coil 436, which allows the battery 428 to be recharged wirelessly. The electronic components 428, 430, 436 are secured within the housing 434, which is positioned around the stain bridge 422 in close proximity to the strain gages 424. The housing 434 may include an outer tube that encases and provides a protective layer around the electronic components. The electronics housing 434 may be made of a non-ferrous material such as PEEK, ABS, PLA or ceramic material that allows RF communication. The electronics package 426 may also have an LED that lights up to indicate information to the user. For example, the LED may signify the instrument 400 is on or off. Alternatively, the LED may indicate when the instrument 400 is measuring a load. Furthermore, the LED may be used to indicate the amount of load by changing color based on the amount force detected.

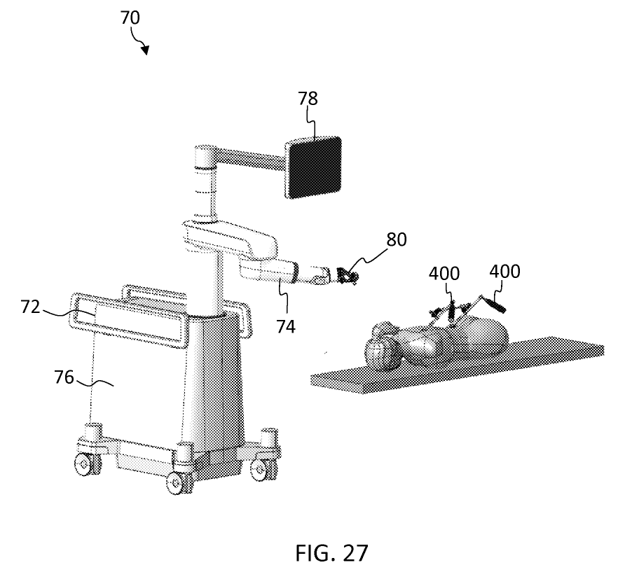

FIG. 26 shows the overall force-sensing rod link reducer instrument system including two rod link reducer instruments 400 and coupling rod 406 connecting the two instruments 400. As previously shown in FIG. 21, the force measuring rod link reducer instruments 400 may be attached to temporary rods 402 secured to pedicle screws 404 placed in the spine. The surgeon holds the handles 412 of the instruments 400 and manipulates the spine to achieve the desired correction. With further reference to FIG. 27, during the procedure, information from the rod link reducers instruments 400 may be communicated to the external computer (e.g., on-board computer for robot system 70). In other words, the strain detected by the strain gages 424 during correction may be directly communicated to the robot system 70 through the wireless communication module. Software receives the strain information and then calculates the forces and moments exerted on the patient. This information may then be used to display relevant information to the user, for example, via screen 78. The information may include actual measurements of force, graphical depictions of relative force, displays of force vectors, warnings about screw pull out or screw plow, or other insights generated from the strain measurements.

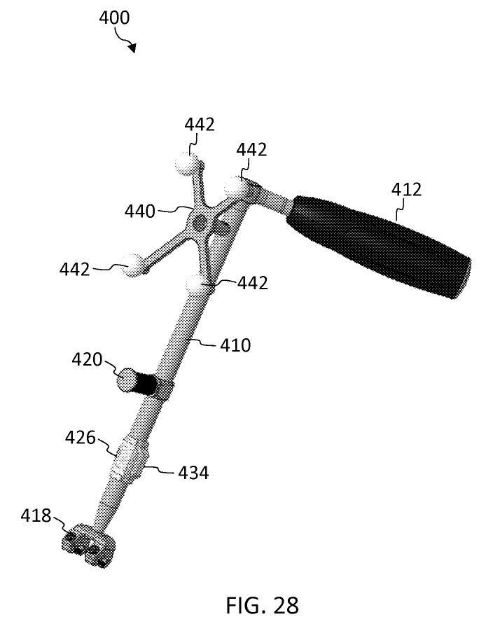

Turning now to FIG. 28, the instrument 400 may be configured to be a navigated force-sensing rod link reducer instrument. In particular, the instrument 400 may further include a tracking array 440 attached to the rod link manipulating arm 410, which is trackable via a navigation system. The array 400 may include reflective spheres 442 or active infrared LEDs that can be tracked by a camera (e.g., camera 84 of the robot system 70) located in the operating room. In other words, the tracking array 440 may be compatible with navigation through robot system 70, 100 or other suitable tracking techniques.

The navigation instrument 400 is configured to measure force exerted on the patient as well as track where the force was applied. This may be especially useful for measuring spine stiffness intraoperatively or for tracking progress during deformity correction. Stiffness may be calculated by dividing force by displacement. The force may be measured by the instrument 400 using the techniques described above while the displacement may be measured by tracking the position of the instrument 400 with the navigation camera 84. The spine stiffness may be calculated by the software and displayed to the surgeon on the screen 78. This allows the surgeon to know when the spine stiffness is low enough to achieve the desired correction intraoperatively, instead of relying entirely on the surgeon’s feelings and judgment.

The additional data may also be used to provide information to the kinematic model of the spine in order to track deformity correction. The kinematic model may define the relationship between each vertebra such that the model can estimate where each vertebra moves in response to a given force or moment input. The starting position of the spine may be known from preoperative imaging such as CT or MRI. The forces and moments applied to the spine may be measured and recorded by the instrument 400 using the methods described herein. The force and moment information may be fed into the software as inputs for the kinematic model. The kinematic model defines the new position of the spine based on the force and moment inputs and defined relationships between vertebrae. The system may then be used to display information to the surgeon intraoperatively, such as intraoperative spinal alignment and progress toward the correction goal. This information may be displayed on the screen 78 in the form of measurements or depictions of the spine in its various alignment states of correction (e.g., preoperative alignment, current alignment, goal alignment).

In one embodiment, the instrument 400 may also include an inertial measurement unit (IMU). The IMU may have three, six, or nine degrees of freedom in order to capture data related to linear acceleration, angular velocity, and orientation. The IMU may allow the system 70, 100 to know the orientation of the instrument 400 in addition to the instrument 400 being able to measure the forces on the spine. By knowing both the amount of force and the orientation of the instrument 400, the software may calculate and display information related to force vectors and moments exerted on the spine. This data may also be used to provide force and moment data to the kinematic model in order to track deformity correction.

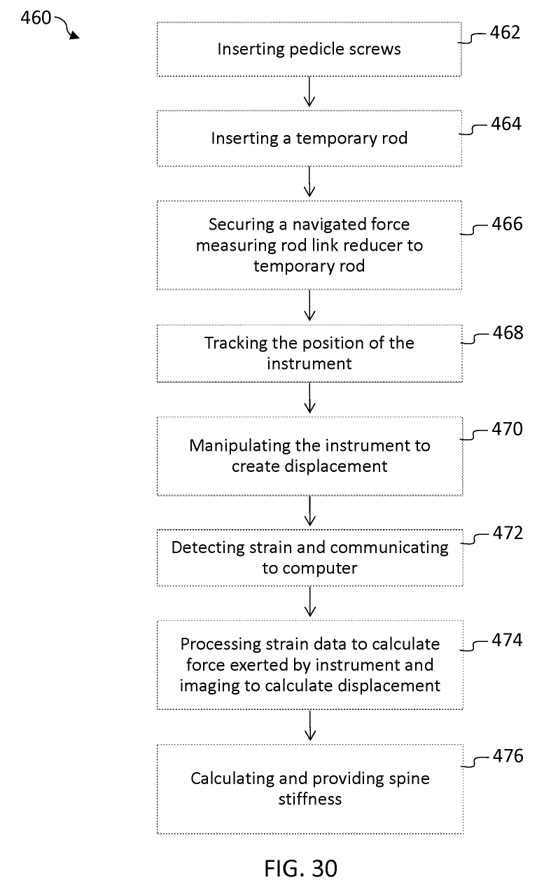

In one embodiment depicted in FIG. 30, a method 460 of assessing spine stiffness intraoperatively may include the following steps. In a first step 462, inserting pedicle screws into the vertebral levels that are to be fixated. In a second step 464, inserting a temporary spinal rod into two or more pedicle screws. In a third step 466, securing the navigated force measuring rod link reducer instrument 400 to the temporary rod. In a fourth step 468, tracking the position of the force measuring rod link reducer instrument 400 with a camera and/or IMU. In a fifth step 470, manipulating the force measuring rod link reducer instrument 400 to create a displacement in the vertebrae fixated by the temporary rod. In a sixth step 472, the force measuring rod link reducer instrument 400 detects the strain signal in the instrument 400 as a result of the applied force and communicates that information to the external computer. In a seventh step 474, the external computer processes the strain data and calculates the force exerted by the instrument as well as processes the navigation data supplied by the camera/IMU to calculate displacement. In an eight step 476, the software calculates the spine stiffness and displays the measurement to the surgeon on a screen. The surgeon may use this additional information to make informed decisions for the surgical procedure in real time.

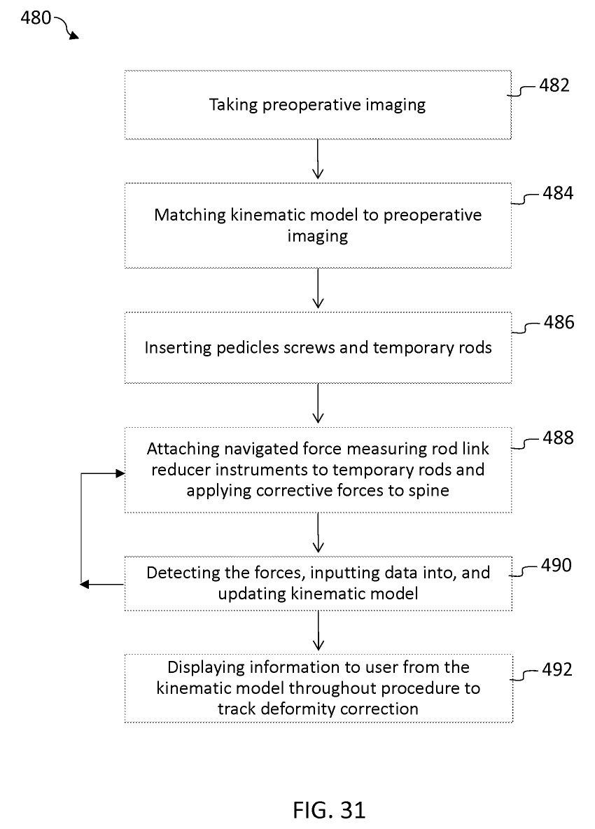

In one embodiment depicted in FIG. 31, a method 480 of tracking deformity correction intraoperatively using a kinematic model of the spine may include the following steps. In a first step 482, preoperative imaging such as fluoroscopy, CT, or MRI may be taken of the patient to determine an initial alignment of the spine. In a second step 484, the initial shape of the kinematic model may be configured to match the initial alignment of the patient’s spine determined from preoperative imaging. The software may be able to measure spinal alignment parameters such as kyphosis, lordosis, and cobb angle from the kinematic model by measuring angles between vertebral endplates of interest. Behavior conditions of the kinematic model may be configured based on patient specific information such as age, weight, sex, bone mineral density, and spine stiffness. This information may be obtained from the patient health record, preoperative assessments, or medical testing. The behavior conditions of the kinematic model may determine how the kinematic model reacts to input variables. For example, spine stiffness may determine how much displacement the kinematic model undergoes for a given input force.

In a third step 486, during surgery, the spine is exposed and the levels to be fixated are instrumented with pedicle screws. Temporary rods may be secured to two or more pedicle screws below the apex of the deformity and two or more pedicle screws above the apex of the deformity. In a fourth step 488, the navigated force measuring rod link reducer instruments 400 may be attached to the temporary rods. The two navigated force measuring rod link reducer instruments 400 may be coupled together with the coupling clamps and coupling rod. The surgeon applies corrective forces to the spine by manipulating the navigated force measuring rod link reducer instruments.

In a fifth step 490, the magnitude, location, and orientation of the forces exerted onto the spine may be detected by the instrument 400 and communicated to and processed by an external computer (e.g., as part of robotic system 70, 100). The software inputs the force magnitude, location, and orientation data into the kinematic model. The kinematic model responds to these inputs by changing shape in such a manner that matches the change in shape of the patient’s actual spine. The kinematic model may update continuously based on force and alignment data measured and provided by the navigated force-sensing rod link reducer instruments 400. In a final step 492, alignment parameters may be continuously calculated from the kinematic model and displayed to the user during surgery to track deformity correction.

The devices and methods for measuring forces exerted on the patient’s spine intraoperatively offer a more accurate method of measuring force instead of subjective assessments which rely on surgeon judgment or speculation. Intraoperative measurements offer surgeons the ability to accurately measure forces during spine surgery, which better inform surgical decision making and increase patient safety. Measurement of spine stiffness may inform the surgeon if osteotomies or ligament releases are needed as well as indicate how much safe correction can be achieved. In addition, measurement of forces placed on implants may prevent implant dislodgement during correction. The measurement devices, systems, and methods may allow for better surgical planning, intraoperative alignment tracking, and safety features related to the correction forces. Leveraging the technological advancements in robotics and navigation may give surgeons enhanced data to optimize clinical outcomes. The spine stiffness data and other relevant information may be aggregated into databases with real time feedback loops for predicting, tracking, and achieving optimal deformity correction for the individual patient.

Although several embodiments of the invention have been disclosed in the foregoing specification, it is understood that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. It is further envisioned that features from one embodiment may be combined or used with the features from a different embodiment described herein. Moreover, although specific terms are employed herein, as well as in the claims which follow, they are used only in a generic and descriptive sense, and not for the purposes of limiting the described invention, nor the claims which follow. The entire disclosure of each patent and publication cited herein is incorporated by reference in its entirety, as if each such patent or publication were individually incorporated by reference herein. Various features and advantages of the invention are set forth in the following claims.

What is claimed is:

1. A method of correcting a spinal deformity of a patient, the method comprising:
applying a force to a spine having a deformity with a force-sensing instrument to measure spine stiffness;
comparing the measured spine stiffness to a database model with existing spinal stiffness parameters;
obtaining guidance from the database model based on patient specific parameters for the patient;
performing a decompression sequentially on the spine, based on the guidance from the database model;
measuring spine stiffness throughout the decompression and updating the database model with each reading on spine stiffness in real time;
obtaining a correction of the deformity when targeted stiffness values are reached based on the database model; and
finalizing the deformity correction by installing spinal hardware.

2. The method of claim 1, wherein the existing spinal stiffness parameters include segmental stiffness, stiffness across motion segments, and/or global stiffness values.

3. The method of claim 1, wherein the existing spinal stiffness parameters include averaged or normalized spine stiffness values.

4. The method of claim 1, wherein the existing spinal stiffness parameters are based on inputs of publicly available data including demographic data and clinical data.

5. The method of claim 4, wherein the clinical data includes spine stiffness data for intact spines, spines having a deformity, and spines having underwent a prior correction.

6. The method of claim 1, wherein the database model includes data aggregated into ranges.

7. The method of claim 1, wherein the force-sensing instrument measures segmental stiffness of a single motion segment between two vertebrae.

8. A method of correcting a spinal deformity of a patient using a robotic surgical system, the method comprising:
providing a robotic surgical system with a robotic arm and a navigation system;
applying a force to a spine having a deformity with a force-sensing instrument coupled to the robotic arm to measure spine stiffness;
comparing the measured spine stiffness to a database model with existing spinal stiffness parameters;
obtaining guidance from the database model based on patient specific parameters for the patient;
performing a decompression sequentially on the spine, based on the guidance from the database model;
measuring spine stiffness throughout the decompression and updating the database model with each reading on spine stiffness in real time;
obtaining a correction of the deformity when targeted stiffness values are reached based on the database model; and
finalizing the deformity correction by installing spinal hardware using the robotic arm.

9. The method of claim 8, wherein the existing spinal stiffness parameters include segmental stiffness, stiffness across motion segments, and/or global stiffness values.

10. The method of claim 8, wherein the existing spinal stiffness parameters include averaged or normalized spine stiffness values.

11. The method of claim 8, wherein the existing spinal stiffness parameters are based on inputs of publicly available data including demographic data and clinical data.

12. The method of claim 11, wherein the clinical data includes spine stiffness data for intact spines, spines having a deformity, and spines having underwent a prior correction.

13. The method of claim 8, wherein the database model includes data aggregated into ranges.

14. The method of claim 8, wherein the force-sensing instrument measures segmental stiffness of a single motion segment between two vertebrae.
